(19) United States
(12) Patent Application Publication
(10) Pub. No.: US 2010/0160361 A1
(43) Pub. Date: Jun. 24, 2010

(54) TREATMENT OF MAJOR ADVERSE CARDIAC EVENTS AND ACUTE CORONARY SYNDROME USING SECRETORY PHOSPHOLIPASE A2 (SPLA2) INHIBITOR OR SPLA2 INHIBITOR COMBINATION THERAPIES

(76) Inventors: Colin Hislop, Menlo Park, CA (US); Joaquim Trias, Millbrae, CA (US); Debra Odink, Oakland, CA (US); Paul Truex, Pleasanton, CA (US)

Correspondence Address: PERKINS COIE LLP, POST OFFICE BOX 1208, SEATTLE, WA, 98111-1208, US

(21) Appl. No.: 12/642,692

(22) Filed: Dec. 18, 2009

Related U.S. Application Data
(60) Provisional application No. 61/139,400, filed on Dec. 19, 2008.
(60) Provisional application No. 61/174,423, filed on Apr. 30, 2009.
(60) Provisional application No. 61/239,967, filed on Sep. 4, 2009.

(51) Int. Cl. A61K 31/404; A61K 31/505; A61K 31/435; A61K 31/47; A61P 9/10; A61P 9/00

(52) U.S. Cl. 514/275; 514/277; 514/311; 514/419

(57) ABSTRACT

Administration of sPLA₂ inhibitors in combination with statins has been found to reduce major adverse cardiac events (MACEs), inflammatory biomarker levels, and LDL-C levels in subjects who have recently experienced an index ACS event to a significantly greater degree than statins alone. These results were unexpected given previous results showing that statins alone are insufficient to satisfactorily reduce MACEs and inflammation in this high-risk population. Therefore, provided herein are methods of treating MACEs, treating ACS, inhibiting inflammation, and lowering cholesterol levels in a subject who has previously experienced an ACS event by administering one or more sPLA₂ inhibitors alone or in combination with one or more statins.

### CROSS-REFERENCE TO RELATED APPLICATIONS

The present application claims the benefit of U.S. Provisional Patent Appl. No. 61/139,400, filed Dec. 19, 2008; U.S. Provisional Patent Appl. No. 61/174,423, filed Apr. 30, 2009; and U.S. Provisional Patent Appl. No. 61/239,967, filed Sep. 4, 2009. The disclosure of each of these applications is incorporated by reference herein in its entirety, including drawings.

### BACKGROUND

In 2004, it was estimated that over 75 million Americans had one or more forms of cardiovascular disease (CVD). Coronary heart disease (CHD) and coronary artery disease (CAD) are the most common types of CVD. CHD and CAD occur when coronary arteries that supply blood to the heart become hardened and narrowed due to plaque build-up along vessel walls (i.e., atherosclerosis). Narrowing of vessel walls in this manner is generally associated with the stable clinical manifestations of atherosclerosis.

Acute manifestations of CVD occur when the atherosclerotic plaque is disrupted, leading to formation of an intracoronary thrombus. Coronary occlusion arising from thrombus formation results in acute coronary syndrome (ACS), a set of ischemic conditions that include unstable angina (UA), non-ST-segment elevation myocardial infarction (NSTEMI), and ST-segment elevation myocardial infarction (STEMI). UA and NSTEMI are generally associated with nonocclusive or partially occlusive thrombus formation, whereas STEM results from a more stable occlusive thrombus. UA and NSTEMI are closely related and have very similar clinical presentations. ACS events affect approximately 1.4 million people in the United States annually as 700,000 new events, 500,000 recurrent events, and 175,000 silent events.

Most of the therapeutic options currently available for treating CHD and CAD function by lowering cholesterol levels, particularly LDL levels. However, many subjects with CHD and CAD do not exhibit elevated cholesterol levels. For example, only 34.1% of men with CHD have hyperlipidemia (Ridker 2005), and half of all myocardial infarctions (MIs) and strokes occur among men and women with LDL levels below recommended thresholds for treatment (Ridker 2008). In addition, CVD is beginning to be viewed not as a simple lipid disease, but also as a complex inflammatory condition. Inflammation contributes to atherosclerotic plaque formation, and also to destabilization of this plaque and subsequent thrombus formation. Thrombus formation is a particular risk in unstable subjects, such as subjects who have recently experienced an ACS event. Existing therapies, which function primarily by lowering cholesterol levels, are insufficient to fully treat CHD and CAD and prevent the events associated with ACS in these populations. Therefore, there is a need in the art for new methods of treating CVD and preventing major adverse cardiac events (MACEs) in unstable populations.

### SUMMARY

In certain embodiments, methods are provided for treating a MACE in a subject in need thereof by administering a therapeutically effective amount of one or more sPLA₂ inhibitors. In certain of these embodiments the one or more sPLA₂ inhibitors include 3-(2-Amino-1,2-dioxoethyl)-2-ethyl-1-(phenylmethyl)-1H-indol-4-yl)oxy)acetic acid (A-001) or a pharmaceutically acceptable salt, solvate, or prodrug thereof. In certain embodiments the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is [[3-(2-Amino-1,2-dioxoethyl)-2-ethyl-1-(phenylmethyl)-1H-indol-4-yl]oxy]acetic acid methyl ester (A-002). In certain embodiments, the subject has previously experienced an ACS event, and in certain of these embodiments the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the MACE is cardiovascular death, fatal or non-fatal MI, UA (including UA requiring urgent hospitalization), fatal or non-fatal stroke, and/or risk of or danger associated with revascularization. In certain embodiments, treatment of a MACE prevents the MACE, reduces the likelihood of occurrence of the MACE, delays the occurrence of the MACE, and/or decreases the severity of the MACE. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, the subject being treated has a condition associated with high baseline inflammation levels, such as diabetes, metabolic syndrome, infection, or autoimmune disease.

In certain embodiments, methods are provided for treating a MACE in a subject in need thereof by administering a therapeutically effective amount of one or more sPLA₂ inhibitors and a therapeutically effective amount of one or more statins. In certain of these embodiments, the one or more sPLA₂ inhibitors include A-001 or a pharmaceutically acceptable salt, solvate, or prodrug thereof. In certain embodiments the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the subject has previously experienced an ACS event, and in certain of these embodiments the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain of these embodiments, administration of one or more sPLA₂ inhibitors in combination with one or more statins treats the MACE more effectively than administration of the one or more statins alone. In certain embodiments, the MACE is cardiovascular death, fatal or non-fatal MI, UA (including UA requiring urgent hospitalization), fatal or non-fatal stroke, and/or risk of or danger associated with revascularization. In certain embodiments, treatment of a MACE prevents the MACE, reduces the likelihood of occurrence of the MACE, delays the occurrence of the MACE, and/or decreases the severity of the MACE. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, the subject being treated has a condition associated with high baseline inflammation levels, such as diabetes, metabolic syndrome, infection, or autoimmune disease.

In certain embodiments, the use of one or more sPLA₂ inhibitors as an adjunct to statin administration to treat a MACE in a subject who has previously experienced an ACS event is provided. In certain of these embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the subject experienced or was diagnosed with the ACS event within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the statin is atorvastatin, rosuvastatin, simvastatin, lovastatin, pravastatin, cerivastatin, fluvastatin, mevastatin, pitavastatin, and/or a statin combination drug. In certain embodiments, treatment of a MACE prevents the MACE, reduces the likelihood of occurrence of the MACE, delays the occurrence of the MACE, and/or decreases the severity of the MACE. In certain of these embodiments, administration of one or more sPLA₂ inhibitors in combination with statin is more effective at preventing MACEs than administration of statin alone. In certain embodiments, the MACE being prevented is cardiovascular death, fatal or non-fatal MI, UA (including UA requiring urgent hospitalization), fatal or non-fatal stroke, and/or risk of or danger associated with revascularization. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, the subject being treated has a condition associated with high baseline inflammation levels, such as diabetes, metabolic syndrome, infection, or autoimmune disease.

In certain embodiments, methods are provided for treating ACS in a subject in need thereof by administering a therapeutically effective amount of one or more sPLA₂ inhibitors. In certain of these embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the subject has previously experienced an ACS event, and in certain of these embodiments the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, the subject being treated has a condition associated with high baseline inflammation levels, such as diabetes, metabolic syndrome, infection, or autoimmune disease.

In certain embodiments, methods are provided for treating ACS in a subject in need thereof by administering a therapeutically effective amount of one or more sPLA₂ inhibitors and a therapeutically effective amount of one or more statins. In certain of these embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the subject has previously experienced an ACS event, and in certain of these embodiments the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain of these embodiments, administration of one or more sPLA₂ inhibitors thereof in combination with one or more statins treats ACS more effectively than administration of the one or more statins alone. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, the subject being treated has a condition associated with high baseline inflammation levels, such as diabetes, metabolic syndrome, infection, or autoimmune disease.

In certain embodiments, methods are provided for inhibiting inflammation in a subject who has previously experienced an ACS event by administering a therapeutically effective amount of one or more sPLA₂ inhibitors. In certain of these embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, administration of one or more sPLA₂ inhibitors reduces levels of one or more inflammatory markers such as hs-CRP, sPLA₂, and/or IL-6. Accordingly, in certain embodiments methods are provided for lowering levels of one or more inflammatory markers by administering one or more sPLA₂ inhibitors. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, administration of one or more sPLA₂ inhibitors results in a decrease in inflammation or in one or more inflammatory marker levels within 12 hours, 24 hours, 36 hours, 48 hours, 4 days, 1 week, 2 weeks, 4 weeks, 8 weeks, or 12 weeks of the first administration. In certain embodiments, the subject being treated has a condition associated with high baseline inflammation levels, such as diabetes, metabolic syndrome, infection, or autoimmune disease.

In certain embodiments, methods are provided for inhibiting inflammation in a subject who has previously experienced an ACS event by administering a therapeutically effective amount of one or more sPLA₂ inhibitors and a therapeutically effective amount of one or more statins. In certain of these embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain embodiments, administration of one or more sPLA₂ inhibitors and one or more statins reduces one or more inflammatory markers such as hs-CRP, sPLA₂, and/or IL-6. Accordingly, in certain embodiments methods are provided for lowering levels of one or more inflammatory markers by administering one or more sPLA₂ inhibitors in combination with one or more statins. In certain embodiments, administration of one or more sPLA₂ inhibitors in combination with one or more statins reduces inflammation and/or levels of one or more inflammatory markers to a greater degree than administration of one or more statins alone. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, administration of one or more sPLA₂ inhibitors and one or more statins results in a decrease in inflammation or in one or more inflammatory marker levels within 12 hours, 24 hours, 36 hours, 48 hours, 4 days, 1 week, 2 weeks, 4 weeks, 8 weeks, or 12 weeks of the first administration. In certain embodiments, the subject being treated has a condition associated with high baseline inflammation levels, such as diabetes, metabolic syndrome, infection, or autoimmune disease.

In certain embodiments, methods are provided for reducing cholesterol levels in a subject who has previously experienced an ACS event by administering a therapeutically effective amount of one or more sPLA₂ inhibitors. In certain embodiments, the one or more sPLA₂ inhibitors include A-001 or a pharmaceutically acceptable salt, solvate, or prodrug. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the reduction in cholesterol levels may include a reduction in total cholesterol, non-HDL cholesterol, and/or LDL-C levels. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, administration of one or more sPLA₂ inhibitors results in a decrease in cholesterol levels within 12 hours, 24 hours, 36 hours, 48 hours, 4 days, 1 week, 2 weeks, 4 weeks, 8 weeks, or 12 weeks of the first administration. In certain embodiments, the subject being treated has a condition associated with high baseline inflammation levels, such as diabetes, metabolic syndrome, infection, or autoimmune disease.

In certain embodiments, methods are provided for reducing cholesterol levels in a subject who has previously experienced an ACS event by administering a therapeutically effective amount of one or more sPLA₂ inhibitors and a therapeutically effective amount of one or more statins. In certain embodiments, the one or more sPLA₂ inhibitors include A-001 or a pharmaceutically acceptable salt, solvate, or prodrug. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain embodiments, the reduction in cholesterol levels may include a reduction in total cholesterol, non-HDL cholesterol, and/or LDL-C levels. In certain embodiments, the reduction in cholesterol observed following administration of the one or more sPLA₂ inhibitors in combination with one or more statins is greater than the reduction observed following administration of the one or more statins alone. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, administration of one or more sPLA₂ inhibitors and one or more statins results in a decrease in cholesterol levels within 12 hours, 24 hours, 36 hours, 48 hours, 4 days, 1 week, 2 weeks, 4 weeks, 8 weeks, or 12 weeks of the first administration. In certain embodiments, the subject being treated has a condition associated with high baseline inflammation levels, such as diabetes, metabolic syndrome, infection, or autoimmune disease.

In certain embodiments, methods are provided for increasing the effectiveness of one or more statins for the treatment of MACEs or ACS by administering a therapeutically effective amount of one or more sPLA₂ inhibitors. In certain embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the MACE being treated is cardiovascular death, fatal or non-fatal MI, UA (including UA requiring urgent hospitalization), fatal or non-fatal stroke, and/or risk of or danger associated with revascularization. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less.

In certain embodiments, methods are provided for increasing the effectiveness of one or more statins for the lowering of cholesterol and/or the reduction of inflammation in a subject who has previously experienced an ACS event by administering a therapeutically effective amount of one or more sPLA₂ inhibitors. In certain embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less. In certain embodiments, the subject has a condition associated with high baseline inflammation levels, such as diabetes, metabolic syndrome, infection, or autoimmune disease.

In certain embodiments, compositions containing one or more sPLA₂ inhibitors are provided for treating MACEs or ACS, lowering cholesterol levels, and/or decreasing inflammation in a subject. In certain embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain of these embodiments, the composition also contains one or more statins, such as for example atorvastatin, rosuvastatin, simvastatin, lovastatin, pravastatin, cerivastatin, fluvastatin, mevastatin, pitavastatin, and/or a statin combination drug. In certain embodiments, the subject has previously experienced an ACS event, and in certain of these embodiments the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the MACE being treated is cardiovascular death, fatal or non-fatal MI, UA (including UA requiring urgent hospitalization), fatal or non-fatal stroke, and/or risk of or danger associated with revascularization. In certain embodiments, the subject being treated has a condition associated with high baseline inflammation levels, such as diabetes, metabolic syndrome, infection, or autoimmune disease.

In certain embodiments, the use of one or more sPLA₂ inhibitors for producing a medicament for use in the treatment of MACEs or ACS, the reduction of cholesterol levels, and/or the reduction of inflammation in a subject is provided. In certain embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, one or more statins are also utilized in producing the medicament. In certain of these embodiments, the one or more statins are atorvastatin, rosuvastatin, simvastatin, lovastatin, pravastatin, cerivastatin, fluvastatin, mevastatin, pitavastatin, and/or a statin combination drug. In certain embodiments, the subject has previously experienced an ACS event, and in certain of these embodiments the ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the MACE being treated is cardiovascular death, fatal or non-fatal MI, UA (including UA requiring urgent hospitalization), fatal or non-fatal stroke, and/or risk of or danger associated with revascularization. In certain embodiments, the subject has a condition associated with high baseline inflammation levels, such as diabetes, metabolic syndrome, infection, or autoimmune disease.

### BRIEF DESCRIPTION OF DRAWINGS

FIG. 1: Effect of A-002 administration on serum LDL levels in ITT population at weeks 2, 4, 8, 16, and 24. ♦=500 mg A-002 plus 80 mg atorvastatin (“A-002”); ▪=80 mg atorvastatin only (“Placebo”).

FIG. 2: Effect of A-002 administration on reaching target LDL levels in ITT population. “A-002” refers to daily administration of 500 mg A-002 and 80 mg atorvastatin, “Placebo” refers to daily administration of 80 mg atorvastatin only. A. % of subjects reaching target LDL level of 70 mg/dl or less. B. % of subjects reaching target LDL level of 70 mg/dl or less.

FIG. 3: Effect of A-002 administration on serum hs-CRP levels in ITT population at weeks 2, 4, 8, 16, and 24. ♦=500 mg A-002 plus 80 mg atorvastatin (“A-002”); ▪=80 mg atorvastatin only (“Placebo”).

FIG. 4: Effect of A-002 administration on serum hs-CRP levels in diabetes subpopulation at weeks 2, 4, 8, 16, and 24. ♦=500 mg A-002 plus 80 mg atorvastatin (“A-002”); ▪=80 mg atorvastatin only (“Placebo”).

FIG. 5: Effect of A-002 administration on serum sPLA₂ levels in ITT population at weeks 2, 4, 8, and 16. ♦=500 mg A-002 plus 80 mg atorvastatin (“A-002”); ▪=80 mg atorvastatin only (“Placebo”).

FIG. 6: Effect of A-002 administration on serum IL-6 levels in ITT population at weeks 2, 4, and 8. ♦=500 mg A-002 plus 80 mg atorvastatin (“A-002”); ▪=80 mg atorvastatin only (“Placebo”).

FIG. 7: Effect of A-002 administration on serum IL-6 levels in diabetes subpopulation at weeks 2, 4, and 8. ♦=500 mg A-002 plus 80 mg atorvastatin (“A-002”); ▪=80 mg atorvastatin only (“Placebo”).

FIG. 8: Effect of A-002 administration on reaching target LDL and CRP levels in ITT population. “A-002” refers to daily administration of 500 mg A-002 and 80 mg atorvastatin, “Placebo” refers to daily administration of 80 mg atorvastatin only. A. % of subjects reaching target LDL level of 70 mg/dl or less and target hs-CRP levels of 3 mg/L or less. B. % of subjects reaching target LDL level of 70 mg/dl or less and target hs-CRP levels of 1 mg/L or less.

FIG. 9: Kaplan-Meier curve showing the percent of subjects in the ITT population experiencing a MACE within 150 days of the first administration of A-002. “A-002” refers to daily administration of 500 mg A-002 and 80 mg atorvastatin, “Placebo” refers to daily administration of 80 mg atorvastatin only.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Abbreviations

ACS, acute coronary syndrome; BMI, body mass index; CAD, coronary artery disease; CHD, coronary heart disease; CK, cardiac troponin; CVD, cardiovascular disease; ECG, electrocardiogram; hs-CRP, high-sensitivity C-reactive protein; LDL or LDL-C, low density lipoprotein; MACE, major adverse cardiac event; MI, myocardial infarction; NSTEMI, non-ST-segment elevation myocardial infarction; sPLA₂, secretory phospholipase A₂; STEM, ST-segment elevation myocardial infarction; t_{1/2}, terminal half-life; TG, triglyceride; UA, unstable angina; ULN, upper limit of normal.

The term “subject” as used herein refers to any mammal, preferably a human.

A “subject in need thereof” refers to a subject currently diagnosed with CVD or exhibiting one or more conditions associated with CVD, a subject who has been diagnosed with or exhibited one or more conditions associated with CVD in the past, or a subject who has been deemed at risk of developing CVD or one or more conditions associated with CVD in the future due to hereditary or environmental factors. In certain embodiments, a subject in need thereof has previously experienced an ACS event, been deemed at risk of experiencing an ACS event, or has exhibited one or more symptoms associated with an ACS event.

“Cardiovascular disease” or “CVD” as used herein includes, for example, atherosclerosis, including coronary artery atherosclerosis and carotid artery atherosclerosis, CAD, CHD, conditions associated with CAD and CHD, cerebrovascular disease and conditions associated with cerebrovascular disease, peripheral vascular disease and conditions associated with peripheral vascular disease, aneurysm, vasculitis, venous thrombosis, diabetes mellitus, and metabolic syndrome.

“Conditions associated with CVD” as used herein include, for example, dyslipidemia, such as for example hyperlipidemia (elevated lipid levels), hypercholesterolemia (elevated cholesterol levels), and hypertriglyceridemia (elevated TG levels), elevated glucose levels, low HDL/LDL ratio, and hypertension.

“Conditions associated with CAD and CHD” as used herein include, for example, ACS.

An “ACS event” or “index ACS event” as used herein refers to UA, NSTEMI, or STEM.

“Angina” as used herein refers generally to chest pain caused by poor blood flow and corresponding decreased oxygen delivery to the heart. Stable or chronic angina occurs only during activity or stress. UA, on the other hand, can occur suddenly without cause. Subjects with angina are at increased risk for MI.

A “major adverse cardiac event” or “MACE” as used herein includes cardiovascular death, fatal or non-fatal MI, UA, fatal or non-fatal stroke, need for a revascularization procedure, heart failure, resuscitated cardiac arrest, and/or new objective evidence of ischemia, as well as any and all subcategories of events falling within each of these event types (e.g., STEM and NSTEMI, documented UA requiring urgent hospitalization). In certain embodiments, MACE refers specifically to cardiovascular death, non-fatal MI, UA requiring urgent hospitalization, non-fatal stroke, and/or need for revascularization procedure.

“Conditions associated with cerebrovascular disease” as used herein include, for example, transient ischemic attack (TIA) and stroke.

“Conditions associated with peripheral vascular disease” as used herein include, for example, claudication.

The term “statin” as used herein refers to any compound that inhibits HMG-CoA reductase, an enzyme that catalyzes the conversion of HMG-CoA to mevalonate.

The terms “treat,” “treating,” or “treatment” as used herein generally refer to preventing a condition or event, slowing the onset or rate of development of a condition or delaying the occurrence of an event, reducing the risk of developing a condition or experiencing an event, preventing or delaying the development of symptoms associated with a condition or event, reducing or ending symptoms associated with a condition or event, generating a complete or partial regression of a condition, lessening the severity of a condition or event, or some combination thereof.

A “reduction” or “decrease” in the level of a particular marker may refer to either a reduction versus baseline or a reduction versus placebo. For example, administration of an sPLA₂ inhibitor may reduce LDL-C levels by dropping LDL-C levels below a previously determined baseline level (e.g., prior to sPLA₂ inhibitor administration or prior to an ACS event). Alternatively, administration of an sPLA₂ inhibitor may reduce LDL-C levels by causing a greater decrease than a placebo at a specific timepoint after administration (e.g., at 1, 2, or 4 weeks after the first administration).

A “reduction in cholesterol levels” as used herein refers to a reduction in total lipoprotein levels and/or a reduction in the level of one or more specific classes of lipoproteins. For example, a reduction in cholesterol levels as used herein may refer to a reduction in one or more of total cholesterol, LDL-C, VLDL, IDL, and non-HDL cholesterol. Similarly, a reduction in LDL-C levels may refer to a reduction in the level of total LDL-C and/or a reduction in the level of one or more subclasses of LDL-C such as LDL-C particles, small LDL-C particles, oxidized LDL-C, and ApoB. A reduction in cholesterol levels may be observed in any biological fluid that normally contains lipoprotein, such as for example serum, blood, or plasma.

With regard to MACEs, the terms “treat,” “treating,” or “treatment” refer to preventing MACEs or MACE recurrence, reducing the likelihood of MACEs or MACE recurrence, delaying the occurrence of MACEs, reducing the severity of MACEs or one or more symptoms associated with MACEs, and/or preventing, delaying or reducing the development of one or more symptoms related to MACEs. For each of these, the effect on MACEs may refer to an effect on MACEs generally (e.g., a reduction in the likelihood of occurrence of all types of MACE), an effect on one or more specific types of MACE (e.g., a reduction in the likelihood of death, non-fatal MI, UA requiring urgent hospitalization, non-fatal stroke, or need for or risk relating to a revascularization procedure), or a combination thereof. In those cases where treatment refers to an effect on one or more specific MACEs, treatment may result in a decrease in the likelihood or severity of one or more types of MACEs without exhibiting an effect on MACEs generally. For example, treatment may result in a shift from a more severe MACE type (e.g., cardiovascular death, fatal MI, or fatal stroke) to a less severe MACE type (e.g., non-fatal MI or non-fatal stroke). In these situations, the likelihood of occurrence of the more severe type of MACE may be decreased without a decrease in MACEs generally, due to a concomitant increase in a less severe type of MACE.

With regard to ACS, the terms “treat,” “treating,” or “treatment” refer to preventing ACS development, advancement, or recurrence, reducing the likelihood of ACS development, advancement, or recurrence, delaying ACS development, advancement, or recurrence, reducing the severity of ACS or one or more symptoms associated with ACS, and/or preventing, delaying, or reducing one or more symptoms associated with ACS. In certain embodiments, treatment of ACS results in a decrease in the likelihood or severity of UA, NSTEMI, and/or STEMI, and/or a decrease in the number or severity of one or more symptoms associated with UA, NSTEMI, and/or STEMI.

A “therapeutically effective amount” of a composition as used herein is an amount of a composition that produces a desired therapeutic effect in a subject, such as treating a target condition. The precise therapeutically effective amount is an amount of the composition that will yield the most effective results in terms of therapeutic efficacy in a given subject. This amount will vary depending upon a variety of factors, including but not limited to the characteristics of the therapeutic composition (including, e.g., activity, pharmacokinetics, pharmacodynamics, and bioavailability), the physiological condition of the subject (including, e.g., age, body weight, sex, disease type and stage, medical history, general physical condition, responsiveness to a given dosage, and other present medications), the nature of the pharmaceutically acceptable carrier or carriers in the composition, and the route of administration. One skilled in the clinical and pharmacological arts will be able to determine a therapeutically effective amount through routine experimentation, namely by monitoring a subject's response to administration of a composition and adjusting the dosage accordingly. For additional guidance, see, e.g., Remington: The Science and Practice of Pharmacy, 21^{st} Edition, Univ. of Sciences in Philadelphia (USIP), Lippincott Williams & Wilkins, Philadelphia, Pa., 2005, and Goodman & Gilman's The Pharmacological Basis of Therapeutics, 11th Edition, McGraw-Hill, New York, N.Y., 2006, the entire disclosures of which are incorporated by reference herein.

A “pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. Such a carrier may comprise, for example, a liquid, gel, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, flavoring agent, or some combination thereof. Each component of the carrier must be “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits. Examples of pharmaceutically acceptable carriers for use in the presently disclosed pharmaceutical compositions include, but are not limited to, diluents such as microcrystalline cellulose or lactose (e.g., anhydrous lactose, lactose fast flo), binders such as gelatin, polyethylene glycol, wax, microcrystalline cellulose, synthetic gums such as polyvinylpyrrolidone, or cellulosic polymers such as hydroxypropyl cellulose (e.g., hydroxypropyl methylcellulose (HPMC)), lubricants such as magnesium stearate, calcium stearate, stearic acid, or microcrystalline cellulose, disintegrants such as starches, cross-linked polymers, or celluloses (e.g., croscarmellose sodium (CCNa), fillers such as silicon dioxide, titanium dioxide, microcrystalline cellulose, or powdered cellulose, surfactants or emulsifiers such as polysorbates (e.g., Polysorbate 20, 40, 60, or 80; Span 20, 40, 60, 65, or 80), antioxidant agents such as butylated hydroxyanisole (BHA), butylated hydroxytoluene (BHT), propyl gallate, or ascorbic acid (either free acid or salt forms thereof), buffers such as phosphate or citrate buffers, sequestering agents such as ethylenediaminetetraacetic acid (EDTA), ethylene glycol tetraacetic acid (EGTA), or edetate disodium, dispersing agents such as sodium carboxymethylcelluose, hydroxypropyl methylcellulose, povidone, or polyvinylpyrrolidone, dissolution aids such as calcium carbonate, and excipients such as water, saline, dextrose, glycerol, or ethanol, citric acid, calcium metabisulfite, lactic acid, malic acid, succinic acid, or tartaric acid.

Reducing cholesterol levels, particularly LDL-C levels, is currently the most common approach for treating CVD and conditions associated therewith. The goal of lowering cholesterol levels is to delay or reverse the onset and progression of atherosclerosis. In stable subjects, vessel narrowing due to atherosclerotic plaque formation is the primary cause of ischemic events such as MI or stroke. Lowering cholesterol levels in these stable subjects prevents additional plaque build-up, thereby reducing the risk and slowing the development CAD and CHD.

Among the most well-known and commonly used compounds for reducing cholesterol levels are statins. Statins inhibit HMG-CoA reductase from catalyzing the conversion of HMG-CoA to mevalonate, a rate-limiting step in the cholesterol biosynthetic pathway. As such, statins inhibit cholesterol biosynthesis and prevent the build-up of arterial plaque. Statin administration has been shown to lower both LDL-C and TG levels, and statins have also been shown to reduce inflammation and decrease blood levels of the inflammatory marker hs-CRP. Statins are routinely administered to stable subjects with chronic hyperlipidemia or established CVD, and have been shown to reduce cardiovascular events to some extent in stable populations with elevated cholesterol levels. In addition, recent studies have shown that statin administration to healthy subjects exhibiting elevated hs-CRP levels without hyperlipidemia lowers LDL-C and hs-CRP levels and decreases the risk of MACEs (Ridker 2008). However, statins are not always effective at preventing cardiovascular events. For example, 60-70% of cardiovascular events continue to occur despite statin therapy (Ridker 2005).

CHD and CAD are no longer viewed simply as lipid diseases, but also as complex inflammatory conditions. Inflammation contributes to atherosclerotic plaque build-up, and also plays a key role in the loss of collagen in the fibrous cap overlying atherosclerotic plaques. This loss of collagen decreases plaque stability, which in turn increases the likelihood of coronary thrombosis, a primary proximate cause of many MACEs. Since reduction of cholesterol levels is insufficient to prevent plaque instability, standard cholesterol-lowering therapies are not necessarily sufficient to treat CHD or CAD.

The danger associated with plaque instability is particularly high in unstable subjects, such as those who have recently experienced an ACS event (e.g., subjects who have experienced one or more ACS events or been diagnosed with one or more ACS events within the past 96 hours). ACS events are following by an acute inflammatory response, which is reflected by a short-term spike in levels of inflammatory markers such as hs-CRP, sPLA₂, and IL-6, as well as a marked decrease in plaque stability. Substantial elevations in sPLA₂ activity are generally observed within 24 hours of an ACS event, and this increased activity can continue for up to 12 weeks after the event. Inflammatory marker levels eventually drop back to pre-ACS event baseline levels, but subjects are at a very high risk of MACEs during the months following the event. LDL levels generally decrease slightly immediately following the event, but this is followed in subsequent weeks by a gradual return to pre-event levels or beyond. During this period, the ideal therapeutic approach is one that rapidly lowers cholesterol levels, prevents or slows a subsequent increase in cholesterol levels, prevents plaque build-up, and restores stability. Statins are routinely administered to the unstable post-ACS event population, but statin therapy alone is insufficient to maintain reduced LDL-C levels and prevent MACEs in these subjects. As patients stabilize, statins are insufficient to entirely prevent the subsequent increase in LDL-C. 15% of subjects who have recently experienced an ACS event and are treated with statin die or experience MI, stroke, or UA within four months after the initial event, and 22% experience these MACEs or require percutaneous coronary intervention (PCI) within two years (Schwartz 2005). Similar therapy data from the PROVE-IT TIMI-22 study demonstrated a 25% recurrent event rate at 2.5 years (Cannon 2004; Ridker 2005). Therefore, there is a need for new therapeutic approaches to treat MACEs and ACS in unstable subjects.

Phospholipases A₂ are a class of enzymes that play a role in inflammation by hydrolyzing the sn-2 fatty acyl chain of glycerophospholipids to produce lysophospholipids, resulting in downstream production of arachidonic acid, prostaglandins, and leukotrienes. The classes of phospholipase A₂ in humans include secretory phospholipase A₂ (sPLA₂) types IB, IIA, IIC, IID, IIE, IIF, III, V, X, and XII, lipoprotein-associated phospholipase A₂ (Lp-PLA₂, also known as PLA₂ type VII), cytosolic phospholipase (cPLA₂), and calcium-independent phospholipase A₂ (iPLA₂). Elevated levels of sPLA₂ types IIA, IID, IIE, IIF, III, V, and X have been observed in all stages of atherosclerosis development and have been implicated in atherogenesis based on their ability to degrade phospholipid (Kimura-Matsumoto 2007). sPLA₂ type IIA has been found to be expressed at vascular smooth muscle cells and foam cells in human arteriosclerosis lesions, and this expression has been correlated to the development of arteriosclerosis (Menschikowski 1995; Elinder 1997; Hurt-Camejo 1997). Transgenic mice that express high levels of human type IIA sPLA₂ have increased LDL-C levels, decreased HDL levels, decreased LDL-C and HDL particle size, and exhibit arteriosclerotic lesions (Ivandic 1999; Tietge 2000), and develop arteriosclerosis at a higher rate compared to normal mice when given a high fat diet (Ivandic 1999). Treatment with sPLA₂ modifies LDL-C lipoproteins such that they have higher affinity for extracellular matrix proteins (Camejo 1998; Sartipy 1999; Hakala 2001), resulting in an increased retention of LDL-C particles in the arterial wall. sPLA₂ treatment also reduces approximately 50% of the phospholipid moiety of normal LDL-C, resulting in smaller and denser particles that are more likely to form non-soluble complexes with proteoglycans and glycosaminoglycans (Sartipy 1999). In addition, there is some evidence that sPLA₂ remodels HDL, resulting in HDL catabolism (Pruzanski 1998). sPLA₂ type V is present in atherosclerotic lesions associated with smooth muscle cells and in surrounding foam cells in lipid core areas of the plaque in mice and humans (Rosengren 2006). sPLA₂ type V has been shown to increase arteriosclerosis in mice, while a deficiency of sPLA₂ type V has been shown to reduce arteriosclerosis (Rosengren 2006; Bostrom 2007). Lp-PLA2 is highly expressed in the necrotic core of coronary lesions (Serruys 2008).

sPLA₂ expression has also been correlated with an increased risk of development of CAD. Higher circulating levels of sPLA₂, and of sPLA₂ type IIA specifically, have been observed in patients with documented CAD than in control patients (Kugiyama 1999; Liu 2003; Boekholdt 2005; Chait 2005; Hartford 2006). In addition, higher circulating levels of sPLA₂ were found to provide an accurate prognostic indicator for development of CAD in healthy individuals (Mallat 2007). Measurement of sPLA₂ activity has been shown to be an independent predictor of death and new or recurrent MI in subjects with ACS, and provides greater prognostic accuracy than measuring type IIA concentration only (Mallat 2005). It has also been proposed that sPLA₂ may have detrimental effects in the setting of ischemic events. This is based largely on the finding of sPLA₂ depositions in the necrotic center of infarcted human myocardium (Nijmeijer 2002).

Previous studies have established that once- or twice-daily administration of the sPLA₂ inhibitor Amino-1,2-dioxoethyl)-2-ethyl-1-(phenylmethyl)-1H-indol-4-yl]oxy]acetic acid methyl ester (A-002) decreases total cholesterol, LDL-C, total LDL-C particle, and small LDL-C particle levels in a stable CVD population, as well as in diabetic and high baseline LDL-C subpopulations (WO2008/137803). In addition, these previous studies established that administration of A-002 in combination with one or more statins results in a synergistic decrease in LDL-C and small LDL-C particle levels in a stable CVD population, including in a high baseline LDL level subpopulation. This effect was not limited to a particular statin, but instead was observed across the entire spectrum of statins. Previous studies have also established that A-002 administration decreases levels of various inflammatory markers such as hs-CRP and sPLA₂ in stable populations.

These previous findings support the use of sPLA₂ inhibitors alone or in combination with other cardiovascular drugs to alter cholesterol levels and treat CVD in a stable population, including subjects with chronically elevated cholesterol levels. However, the ability to lower cholesterol levels and treat CVD in a stable population does not necessarily correlate with the ability to rapidly lower cholesterol levels and reduce MACEs in an unstable population, such as a population that has recently suffered an ACS event. As discussed above, the acute inflammatory response following an ACS event places these unstable subjects at very high risk of MACEs. For this reason, therapeutics that successfully lower cholesterol levels and decrease MACEs in stable populations have proven to be less successful in unstable ACS populations. For example, one study examining the effect of 80 mg atorvastatin administration in subjects that had recently experienced an ACS event found only a 2.6% absolute reduction and a 16% relative reduction in death, non-fatal MI, cardiac arrest with resuscitation, or worsening symptomatic myocardial ischemia (Schwartz 2001). Therefore, there is a need for novel therapies to prevent MACE occurrence and lower cholesterol levels in a prompt manner in acute post-ACS populations.

As disclosed herein, administration of A-002 to an unstable population that has recently experienced an ACS event significantly reduces inflammation (as evidenced by decreases in mean and median levels of the inflammatory markers hs-CRP, sPLA₂, and IL-6). Importantly, this improvement in inflammatory marker levels was observed as early as week 2, the first timepoint measured. All subjects in the trial were simultaneously receiving statin, the standard therapy for post-ACS subjects. Therefore, the results disclosed herein establish that administration of an sPLA₂ inhibitor in combination with statin significantly shortens the period of acute inflammation following an ACS event.

As discussed above, hs-CRP, sPLA₂, and IL-6 levels spike immediately after an ACS event, and then slowly return to pre-event baselines. Since this initial spike is associated with a greatly increased risk of MACEs, the ability to reduce inflammation as quickly as possible after the ACS event is key to MACE reduction. The difference in inflammatory marker levels between the A-002/statin and statin subpopulations became less marked at later timepoints, but subjects receiving the A-002/statin combination continued to exhibit greater decreases in inflammatory marker levels than subjects receiving statin only. Therefore, the combination of sPLA₂ inhibitor and statin continues to reduce inflammation in the later weeks following an ACS event.

Administration of A-002 also significantly lowered inflammatory marker levels in a diabetic subpopulation that had recently experienced an ACS event. This is important because it establishes that A-002 in combination with statin is capable of decreasing inflammation in a population that is particularly vulnerable to cardiovascular disease due to high baseline inflammation levels. These results suggest that A-002 plus statin will lower inflammation in other post-ACS event populations with marked levels of baseline inflammation, such as subjects with metabolic syndrome.

The results disclosed herein further show that administration of A-002 to an unstable population that has recently experienced an ACS event significantly lowers LDL-C levels. As with the inflammatory markers, the reduction in LDL-C was observed as early as week 2, the first timepoint measured. The difference in cholesterol levels between the A-002/statin and statin subpopulations became less marked at later timepoints, but subjects receiving the A-002/statin combination continued to exhibit greater decreases in cholesterol levels than subjects receiving statin only. As discussed above, LDL levels tend to decrease slightly immediately following an ACS event, followed by a gradual increase to pre-event levels in subsequent weeks. The results disclosed herein establish that administration of an sPLA₂ inhibitor in combination with statin not only causes LDL levels to drop more rapidly than normal immediately following an ACS event, but also maintains low LDL levels over the ensuing weeks and months.

As further disclosed herein, administration of A-002 and statin decreased MACEs to a greater extent than statin alone over a time period of 16 weeks. As expected in a post-ACS event population, the majority of MACEs occurred during the first 90 days following the index ACS event, with most occurring during the first 30 days. During this critical time period, A-002 significantly decreased the number of MACEs. The decrease in MACEs following A-002 administration was observed across a range of MACE types, including UA requiring urgent hospitalization, MI, and death. In addition to reducing the number of MACEs following an ACS event, administration of A-002 may decrease the severity of MACEs.

Based on the results disclosed herein, methods are provided for treating MACEs, including reducing the likelihood of MACEs, treating ACS, reducing inflammation, reducing blood levels of one or more inflammatory markers such as hs-CRP, sPLA₂, or IL-6, and treating dyslipidemia (including lowering non-HDL cholesterol, LDL-C, and/or total cholesterol levels) in a subject who has previously experienced an ACS event or has been deemed at risk of suffering an ACS event by administering a therapeutically effective amount of one or more PLA₂ inhibitors alone or in combination with one or more therapeutics used in the treatment of MACEs or ACS. In certain embodiments, the one or more PLA₂ inhibitors are selected from sPLA₂, Lp-PLA₂, and cPLA₂ inhibitors, and in certain of these embodiments one or more of the PLA₂ inhibitors are sPLA₂ inhibitors. In certain embodiments, the one or more therapeutics used in the treatment of MACEs or ACS include one or more statins. Further provided herein are compositions, products, and pharmaceutical formulations comprising one or more PLA₂ inhibitors alone or in combination with one or more therapeutics used in the treatment of MACEs or ACS, as well as the use of one or more PLA₂ inhibitors alone or in combination with one or more MACE or ACS therapeutics to create a medicament for use in the methods disclosed herein.

In certain embodiments, an sPLA₂ inhibitor for use in the methods and compositions disclosed herein may be an indole-based sPLA₂ inhibitor, meaning that the compound contains an indole nucleus having the structure:

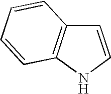

A variety of indole-based sPLA₂ inhibitors are known in the art. For example, indole-based sPLA₂ inhibitors that may be used in conjunction with the present invention include but are not limited to those set forth in U.S. Pat. No. 5,654,326 (Bach); U.S. Pat. No. 5,733,923 (Bach); U.S. Pat. No. 5,919,810 (Bach); U.S. Pat. No. 5,919,943 (Bach); U.S. Pat. No. 6,175,021 (Bach); U.S. Pat. No. 6,177,440 (Bach); U.S. Pat. No. 6,274,578 (Denney); and U.S. Pat. No. 6,433,001 (Bach), the entire disclosures of which are incorporated by reference herein. Methods of making indole-based sPLA₂ inhibitors are set forth in, for example, U.S. Pat. Nos. 5,986,106 (Khau); U.S. Pat. No. 6,265,591 (Anderson); and U.S. Pat. No. 6,380,397 (Anderson), the entire disclosures of which are incorporated by reference herein. sPLA₂ inhibitors for use in the present invention may be generated using these synthesis methods, or using any other synthesis method known in the art. In certain embodiments, sPLA₂ inhibitors for use in the present invention may be sPLA₂ type IIA, type V, and/or type X inhibitors. Various examples of indole-based sPLA₂ inhibitors are set forth below. These examples are merely provided as illustrations of the types of inhibitors that may be used in conjunction with the methods and compositions disclosed herein, and as such are not meant to be limiting. One of ordinary skill in the art will recognize that a variety of other indole-based sPLA₂ inhibitors may be used.

In certain embodiments, sPLA₂ inhibitors for use in the current invention are 1H-indole-3-glyoxylamide compounds having the structure:

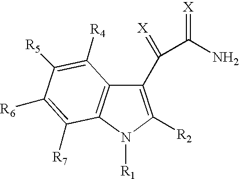

wherein:
each X is independently oxygen or sulfur;
R₁ is selected from the group consisting of (a), (b), and (c), wherein:

(a) is C₇-C₂₀ alkyl, C₇-C₂₀ alkenyl, C₇-C₂₀ alkynyl, carbocyclic radicals, or heterocyclic radicals;

(b) is a member of (a) substituted with one or more independently selected non-interfering substituents; and

(c) is the group -(L)-R₈₀, where, -(L)- is a divalent linking group of 1 to 12 atoms selected from carbon, hydrogen, oxygen, nitrogen, and sulfur, wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) carbon, hydrogen, and oxygen only; and where R₈₀ is a group selected from (a) or (b);
R₂ is hydrogen, halo, C₁-C₃ alkyl, C₃-C₄ cycloalkyl, C₃-C₄ cycloalkenyl, —O—(C₁-C₂ alkyl, —S—(C₁-C₂ alkyl), or a non-interfering substitute having a total of 1 to 3 atoms other than hydrogen;
R₄ and R₅ are independently selected from the group consisting of hydrogen, a non-interfering substituent, and -(Lₐ)-(acidic group), wherein -(Lₐ)- is an acid linker having an acid linker length of 1 to 4; provided that at least one of R₄ and R₅ must be -(Lₐ)- (acidic group);
R₆ and R₇ are each independently selected from hydrogen, non-interfering substituents, carbocyclic radicals, carbocyclic radicals substituted with non-interfering substituents, heterocyclic radicals, and heterocyclic radicals substituted with non-interfering substituents;
provided that for any of the groups R₁, R₆, and R₇, the carbocyclic radical is selected from the group consisting of cycloalkyl, cycloalkenyl, phenyl, naphthyl, norbornanyl, bicycloheptadienyl, tolulyl, xylenyl, indenyl, stilbenyl, terphenylyl, diphenylethylenyl, phenyl-cyclohexenly, acenaphthylenyl, and anthracenyl, biphenyl, bibenzylyl and related bibenzylyl homologues represented by the formula (bb),

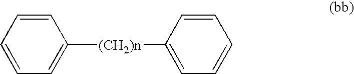

where n is a number from 1 to 8; provided, that for any of the groups R₁, R₆, and R₇, the heterocyclic radical is selected from the group consisting of pyrrolyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, thianaphtheneyl, dibenzothiophenyl, indazolyl, imidazo(1.2-A)pyridinyl, benzotriazolyl, anthranilyl, 1,2-benzisoxazolyl, benzoxazolyl, benzotriazolyl, purinyl, pryidinyl, dipyridylyl, phenylpyridinyl, benzylpyridinyl, pyrimidinyl, phenylpyrimidinyl, pyrazinyl, 1,3,5-triazinyl, quinolinyl, phthalazinyl, quinazolinyl, and quinoxalinyl; and
provided that for the groups R₁, R₂, R₄, R₅, R₆, and R₇ the non-interfering substituent is selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl, where n is from 1 to 8;
and pharmaceutically acceptable salts, solvates, prodrug derivatives, racemates, tautomers, or optical isomers thereof.

In certain of these embodiments, -(L)- has the formula:

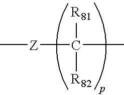

wherein R₈₁ and R₈₂ are each independently selected from the group consisting of hydrogen, C₁-C₁₀ alkyl, carboxy, carbalkoxy, and halo; p is a number from 1 to 5; and Z is selected from the group consisting of a bond, —(CH₂)—, —O—, —N(C₁-C₁₀ alkyl)-, —NH—, and —S—.

In certain of these embodiments wherein R₄ is -(Lₐ)-(acidic group), the acid linker -(Lₐ)- has the formula:

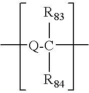

wherein Q is selected from the group consisting of —(CH₂)—, —O—, —NH—, and —S—; and R₈₃ and R₈₄ are each independently selected from the group consisting of hydrogen, C₁-C₁₀ alkyl, aryl, C₁-C₁₀ alkaryl, C₁-C₁₀ aralkyl, hydroxy, and halo.

In certain of these embodiments wherein R₅ is -(Lₐ)-(acidic group), the acid linker -(Lₐ)- has the formula:

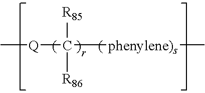

wherein r is a number from 2 to 7; s is 0 or 1; Q is selected from the group consisting of —(CH₂)—, —O—, —NH—, and —S—; and R₈₅ and R₈₆ are each independently selected from the group consisting of hydrogen, C₁-C₁₀ alkyl, aryl, C₁-C₁₀ alkaryl, C₁-C₁₀ aralkyl, carboxy, carbalkoxy, and halo.

In certain embodiments, a 1H-indole-3-glyoxylamide compound for use in the present invention is selected from the group consisting of: ((3-(2-Amino-1,2-dioxoethyl)-2-ethyl-1-(phenylmethyl)-1H-indol-4-yl)oxy)acetic acid; [[3-(2-Amino-1,2-dioxoethyl)-2-ethyl-1-(phenylmethyl)-1H-indol-4-yl]oxy]acetic acid methyl ester; ((3-(2-Amino-1,2-dioxoethyl)-2-methyl-1-(phenylmethyl)-1H-indol-4-yl)oxy)acetic acid; dl-2-((3-(2-Amino-1,2-dioxoethyl)-2-methyl-1-(phenylmethyl)-1H-indol-4-yl)oxy)propanoic acid; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-(1,1′-biphenyl)-3-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-1-(3-chlorophenyl)methyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid; ((3-(2-Amino-1,2-dioxoethyl)-2-cyclopropyl-1-(phenylmethyl)-1H-indol-4-yl) oxy)acetic acid; ((3-(2-Amino-1,2-dioxoethyl)-1-(1,1′-biphenyl)-2-ylmethyl)-2-cyclopropyl-1H-indol-4-yl)oxy)acetic acid; and 4-((3-(2-Amino-1,2-dioxoethyl)-2-ethyl-1-(phenylmethyl)-1H-indol-5-yl)oxy) butanoic acid, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

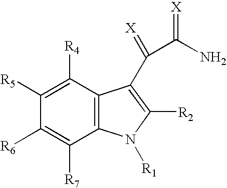

wherein:
both X are oxygen;
R₁ is selected from the group consisting of:

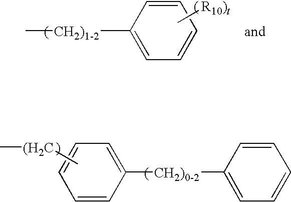

wherein R₁₀ is a radical independently selected from halo, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently, selected from the group consisting of hydrogen, a non-interfering substituent, and -(Lₐ)-(acidic group), wherein -(Lₐ)- is an acid linker;
provided that the acid linker -(Lₐ)- for R₄ is selected from the group consisting of:

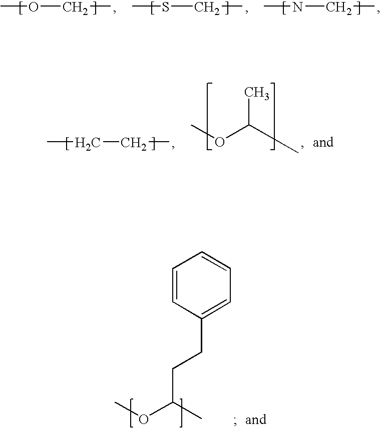

provided that the acid linker -(Lₐ)- for R₅ is selected from the group consisting of:

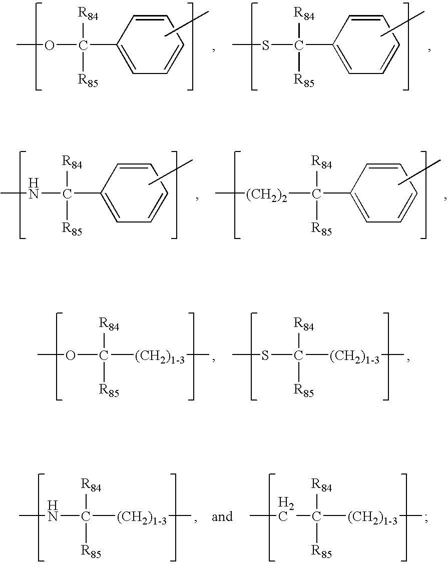

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ alkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, 1H-indole-3-glyoxylamide compounds for use in the present invention are selected from the group consisting of: ((3-(2-Amino-1,2-dioxoethyl)-2-methyl-1-(phenylmethyl)-1H-indol-4-yl)oxy)acetic acid; ((3-(2-Amino-1,2-dioxoethyl)-2-methyl-1-(phenylmethyl)-1H-indol-4-yl)oxy)acetic acid methyl ester; dl-2-((3-(2-Amino-1,2-dioxoethyl)-2-methyl-1-(phenylmethyl)-1H-indol-4-yl) oxy)propanoic acid; dl-2-((3-(2-Amino-1,2-dioxoethyl)-2-methyl-1-(phenylmethyl)-1H-indol-4-yl) oxy)propanoic acid methyl ester; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-2-ylmethyl)-2-methyl-1H-indol-4-yl)oxy)acetic acid methyl ester; ((3-(2-Amino-1,2-dioxoethyl)-1-((1,1′-biphenyl)-3-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid methyl ester; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid methyl ester; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid methyl ester; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid methyl ester; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid methyl ester; ((3-(2-Amino-1,2-dioxoethyl)-1-((3-chlorophenyl)methyl)-2-ethyl-1H-indol-4-yl)oxy)acetic acid; ((3-(2-Amino-1,2-dioxoethyl)-1-((3-chlorophenyl)methyl)-2-ethyl-1H-indol-4-yl)oxy)acetic acid methyl ester; ((3-(2-Amino-1,2-dioxoethyl)-1-((1,1′-biphenyl)-2-ylmethyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid methyl ester; ((3-(2-amino-1,2-dioxoethyl)-1-((1,1′-biphenyl)-2-ylmethyl)-2-propyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid methyl ester; ((3-(2-Amino-1,2-dioxoethyl)-2-cyclopropyl-1-(phenylmethyl)-1H-indol-4-yl) oxy)acetic acid; ((3-(2-Amino-1,2-dioxoethyl)-2-cyclopropyl-1-(phenylmethyl)-1H-indol-4-yl) oxy)acetic acid methyl ester; ((3-(2-Amino-1,2-dioxoethyl)-1-((1,1′-biphenyl)-2-ylmethyl)-2-cyclopropyl-1H-indol-4-yl)oxy)acetic acid; ((3-(2-Amino-1,2-dioxoethyl)-1-((1,1′-biphenyl)-2-ylmethyl)-2-cyclopropyl-1H-indol-4-yl)oxy)acetic acid methyl ester; 4-((3-(2-Amino-1,2-dioxoethyl)-2-ethyl-1-(phenylmethyl)-1H-indol-5-yl)oxy) butanoic acid; 4-((3-(2-Amino-1,2-dioxoethyl)-2-ethyl-1-(phenylmethyl)-1H-indol-5-yl)oxy) butanoic acid tert-butyl ester, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

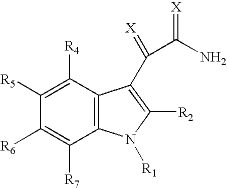

wherein:
each X is independently oxygen or sulfur;
R₁ is selected from groups (a), (b), and (c) wherein:

(a) is C₇-C₂₀ alkyl, C₇-C₂₀ alkenyl, C₇-C₂₀ alkynyl, carbocyclic radical, or heterocyclic radical;

(b) is a member of (a) substituted with one or more independently selected non-interfering substituents; and

(c) is the group -(L)-R₈₀, wherein -(L)- is a divalent linking group of 1 to 12 atoms selected from carbon, hydrogen, oxygen, nitrogen, and sulfur; wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) and carbon, hydrogen, and oxygen only; and where R₈₀ is a group selected from (a) or (b);
R₂ is selected from the group consisting of hydrogen, halo, C₁-C₃ alkyl, C₃-C₄ cycloalkyl, C₃-C₄ cycloalkenyl, —O—(C₁-C₂ alkyl), —S—(C₁-C₂ alkyl), and a non-interfering substituent having a total of 1 to 3 atoms other than hydrogen;
R₄ and R₅ are independently selected from the group consisting of hydrogen, a non-interfering substituent, and the group -(Lₐ)-(acidic group), wherein -(Lₐ)- is an acid linker having an acid linker length of 1 to 4; provided that at least one of R₄ and R₅ is -(Lₐ)-(acidic group);
R₆ and R₇ are each independently selected from the group consisting of hydrogen, non-interfering substituents, carbocyclic radicals, carbocyclic radicals substituted with non-interfering substituents, heterocyclic radicals, and heterocyclic radicals substituted with non-interfering substituents;
and pharmaceutically acceptable salts, solvates, prodrug derivatives, racemates, tautomers, or optical isomers thereof.

In certain embodiments, sPLA₂ inhibitors for use in the current invention are methyl ester prodrug derivatives of 1H-indole-3-glyoxylamide compounds having the structure:

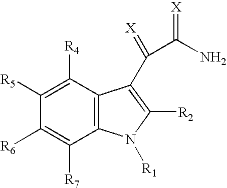

wherein:
both X are oxygen;
R₁ is selected from the group consisting of:

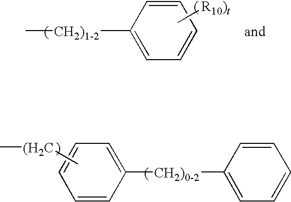

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker;
provided that the acid linker -(Lₐ)- for R₄ is selected from the group consisting of:

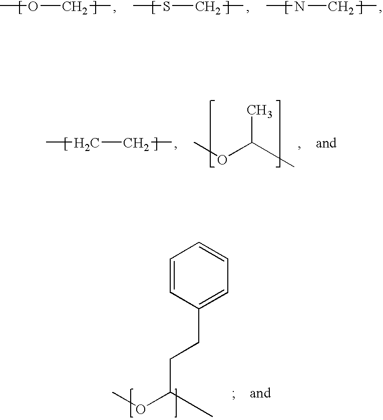

provided that the acid linker -(Lₐ)- for R₅ is selected from the group consisting of:

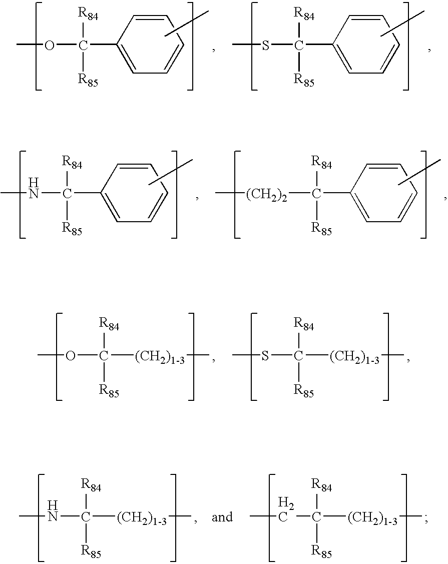

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are (acyloxy) alkyl ester prodrug derivatives of 1H-indole-3-glyoxylamide compounds having the structure:

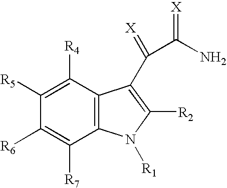

wherein:
both X are oxygen;
R₁ is selected from the group consisting of:

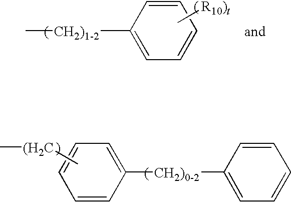

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker;
provided that the acid linker -(Lₐ)- for R₄ is selected from the group consisting of:

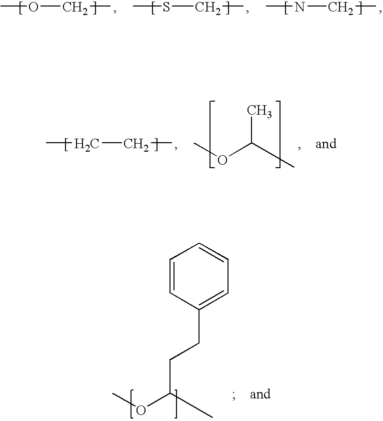

provided that the acid linker -(Lₐ)- for R₅ is selected from the group consisting of:

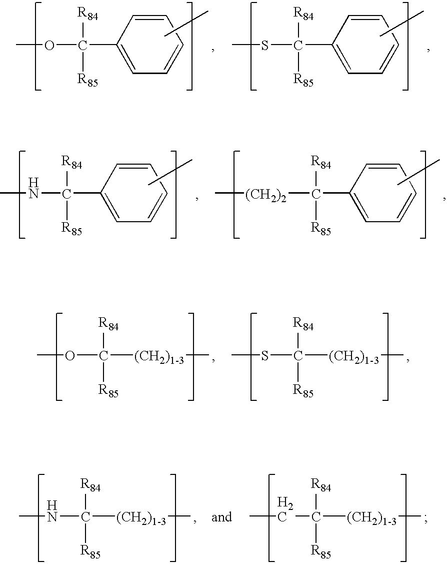

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

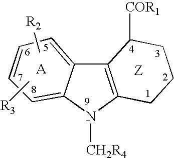

wherein:
R₁ is selected from the group consisting of —NHNH₂ and —NH₂;
R₂ is selected from the group consisting of —OH and —O(CH₂)ₘR₅; wherein R₅ is selected from the group consisting of H, —CO₂H, —CO₂(C₁-C₄ alkyl), —SO₃H, SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

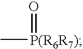

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ is selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl;
wherein R₉ and R₁₀ are each independently —(C₁-C₄)alkyl or -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, and phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy, or naphthyl;
A is selected from the group consisting of phenyl and pyridyl wherein the nitrogen is at the 5-, 6-, 7-, or 8-position;
Z is selected from the group consisting of cyclohexenyl, phenyl, pyridyl wherein the nitrogen is at the 1-, 2-, or 3-position, and a 6-membered heterocyclic ring having one heteroatom selected from the group consisting of sulfur and oxygen at the 1-, 2-, or 3-position and nitrogen at the 1-, 2-, 3-, or 4-position, or wherein one carbon on the heterocyclic ring is optionally substituted with ═O; and wherein one of A or Z is a heterocyclic ring;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

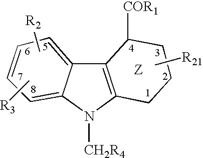

wherein:
Z is selected from the group consisting of cyclohexenyl and phenyl;
R₂₁ is a non-interfering substituent;
R₁ is —NHNH₂ or —NH₂;
R₂ is selected from the group consisting of —OH and —O(CH₂)ₘR₅; wherein R₅ is selected from the group consisting of H, —CO₂H, —CONH₂, —CO₂(C₁-C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

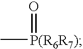

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl; R₉ and R₁₀ are each independently selected from the group consisting of H, —CF₃, phenyl, —(C₁-C₄)alkyl, —(C₁-C₄)alkylphenyl, and -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, phenyl substituted with —(C₁-C₆)alkyl, halo, —CF₃, —OCF₃, —(C₁-C₄)alkoxy, —CN, —(C₁-C₄)alkylthio, -phenyl(C₁-C₄)alkyl, —(C₁-C₄)alkylphenyl, phenyl, phenoxy and naphthyl;
and pharmaceutically acceptable salts, solvates, prodrug derivatives, racemates, tautomers, or optical isomers thereof.

In certain embodiments, sPLA₂ inhibitors for use in the current invention are selected from the group consisting of: {9-[(phenyl)methyl]-5-carbamoylcarbazol-4-yl}oxyacetic acid; 9-benzyl-5,7-dimethoxy-1,2,3,4-tetrahydrocarbazole-4-carboxylic acid hydrazide; 9-benzyl-5,7-dimethoxy-1,2,3,4-tetrahydrocarbazole-4-carboxamide; [9-benzyl-4-carbamoyl-7-methoxy-1,2,3,4-tetrahydrocarbazol-5-yl]oxyacetic acid; [9-benzyl-4-carbamoyl-7-methoxycarbazol-5-yl]oxyacetic acid; methyl [9-benzyl-4-carbamoyl-7-methoxycarbazol-5-yl]oxyacetic acid; 9-benzyl-7-methoxy-5-cyanomethyloxy-1,2,3,4-tetrahydrocarbazole-4-carboxamide; 9-benzyl-7-methoxy-5-(1H-tetrazol-5-yl-methyl)oxy)-1,2,3,4-tetrahydrocarbazole-4-carboxamide; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazol-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-4-carbamoyl-8-chloro-1,2,3,4-tetrahydrocarbazol-5-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; {9-[(3-fluorophenyl)methyl]-5-carbamoylcarbazol-4-yl}oxyacetic acid; {9-[(3-chlorophenyl)methyl]-5-carbamoylcarbazol-4-yl}oxyacetic acid; {9-[(3-phenoxyphenyl)methyl]-5-carbamoylcarbazol-4-yl}oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(3-trifluoromethylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(2-methylphenyl)methyl]-5-carbamoylcarbazol-4-yl}oxyacetic acid; {9-[(3-methylphenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(3-trifluoromethoxyphenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazole-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-5-carbamoyl-1-fluorocarbazol-4-yl]oxyacetic acid; [9-benzyl-4-carbamoyl-8-chloro-1,2,3,4-tetrahydrocarbazol-5-yl]oxyacetic acid; [9-benzyl-5-carbamoyl-1-chlorocarbazol-4-yl]oxyacetic acid; [9-[(Cyclohexyl)methyl]-5-carbamoylcarbazol-4-yl]oxyacetic acid; [9-[(Cyclopentyl)methyl]-5-carbamoylcarbazol-4-yl]oxyacetic acid; [5-carbamoyl-9-(phenylmethyl)-2-(2-thienyl)carbazol-4-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; (R,S)-(9-benzyl-4-carbamoyl-1-oxo-3-thia-1,2,3,4-tetrahydrocarbazol-5-yl)oxyacetic acid; (R,S)-(9-benzyl-4-carbamoyl-3-thia-1,2,3,4-tetrahydrocarbazol-5-yl)oxyacetic acid; 2-(4-oxo-5-carboxamido-9-benzyl-9H-pyrido[3,4-b]indolyl)acetic acid chloride; [N-benzyl-1-carbamoyl-1-aza-1,2,3,4-tetrahydrocarbazol-8-yl]oxyacetic acid; 4-methoxy-6-methoxycarbonyl-10-phenylmethyl-6,7,8,9-tetrahydropyrido[1,2-a]indole; (4-carboxamido-9-phenylmethyl-4,5-dihydrothiopyrano[3,4-b]indol-5-yl)oxyacetic acid; 3,4-dihydro-4-carboxamidol-5-methoxy-9-phenylmethylpyrano[3,4-b]indole; 2-[(2,9 bis-benzyl-4-carbamoyl-1,2,3,4-tetrahydro-betacarbolin-5-yl)oxy]acetic acid; 2-[4-oxo-5-carboxamido-9-(2-methylbenzyl)-9H-pyrido[3,4-b]indolyl]acetic acid; 2-[4-oxo-5-carboxamido-9-(3-methylbenzyl)-9H-pyrido[3,4-b]indolyl]acetic acid; 2-[4-oxo-5-carboxamido-9-(4-methylbenzyl)-9H-pyrido[3,4-b]indolyl]acetic acid; 2-[4-oxo-5-carboxamido-9-(4-tert-butylbenzyl)-9H-pyrido[3,4-b]indolyl]acetic acid; 2-[4-oxo-5-carboxamido-9-pentafluorobenzyl-9H-pyrido[3,4-b]indolyl]acetic acid; 2-[4-oxo-5-carboxamido-9-(2-fluorobenzyl)-9H-pyrido[3,4-b]indolyl]acetic acid; 2-[4-oxo-5-carboxamido-9-(3-fluorobenzyl)-9H-pyrido[3,4-b]indolyl]acetic acid; 2-[4-oxo-5-carboxamido-9-(4-fluorobenzyl)-9H-pyrido[3,4-b]indolyl]acetic acid; 2-[4-oxo-5-carboxamido-9-(2,6-difluorobenzyl)-9H-pyrido[3,4-b]indolyl]acetic acid; 2-[4-oxo-5-carboxamido-9-(3,4-difluorobenzyl)-9H-pyrido[3,4-b]indolyl]acetic acid; 2-[4-oxo-5-carboxamido-9-(2,5-difluorobenzyl)-9H-pyrido[3,4-b]indolyl]acetic acid; 2-[4-oxo-5-carboxamido-9-(3,5-difluorobenzyl)-9H-pyrido[3,4-b]indolyl]acetic acid; 2-[4-oxo-5-carboxamido-9-(2,4-difluorobenzyl)-9H-pyrido[3,4-b]indolyl]acetic acid; 2-[4-oxo-5-carboxamido-9-(2,3-difluorobenzyl)-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[3-(trifluoromethyl)benzyl]-9H-pyrido[3,4-b]indolyl]acetic acid; 2-[4-oxo-5-carboxamido-9-[4-(trifluoromethyl)benzyl]-9H-pyrido[3,4-b]indolyl]acetic acid; 2-[4-oxo-5-carboxamido-9-[3,5-bis(trifluoromethyl)benzyl]-9H-pyrido[3,4-b]indolyl]acetic acid; 2-[4-oxo-5-carboxamido-9-[2,4-bis(trifluoromethyl)benzyl]-9H-pyrido[3,4-b]indolyl]acetic acid; 2-[4-oxo-5-carboxamido-9-(a-methylnaphthyl)-9H-pyrido[3,4-b]indolyl]acetic acid; 2-[4-oxo-5-carboxamido-9-(b-methylnaphthyl)-9H-pyrido[3,4-b]indolyl]acetic acid; 2-[4-oxo-5-carboxamido-9-(3,5-dimethylbenzyl)-9H-pyrido[3,4-b]indolyl]acetic acid; 2-[4-oxo-5-carboxamido-9-(2,4-dimethylbenzyl)-9H-pyrido[3,4-b]indolyl]acetic acid; 2-[4-oxo-5-carboxamido-9-(2-phenylbenzyl)-9H-pyrido[3,4-b]indolyl]acetic acid; 2-[4-oxo-5-carboxamido-9-(3-phenylbenzyl)-9H-pyrido[3,4-b]indolyl]acetic acid; 2-[4-oxo-5-carboxamido-9-(4-phenylbenzyl)-9H-pyrido[3,4-b]indolyl]acetic acid; 2-[4-oxo-5-carboxamido-9-(1-fluorenylmethy)-9H-pyrido[3,4-b]indolyl]acetic acid; 2-[4-oxo-5-carboxamido-9-(2-fluoro-3-methylbenzyl)-9H-pyrido[3,4-b]indolyl]acetic acid; 2-[4-oxo-5-carboxamido-9-(3-benzoylbenzyl)-9H-pyrido[3,4-b]indolyl]acetic acid; 2-[4-oxo-5-carboxamido-9-(2-phenoxybenzyl)-9H-pyrido[3,4-b]indolyl]acetic acid; 2-[4-oxo-5-carboxamido-9-(3-phenoxybenzyl)-9H-pyrido[3,4-b]indolyl]acetic acid; 2-[4-oxo-5-carboxamido-9-(4-phenoxybenzyl)-9H-pyrido[3,4-b]indolyl]acetic acid; 2-[4-oxo-5-carboxamido-9-[3-[2-(fluorophenoxy)benzyl]]-9H-pyrido[3,4-b]indolyl]acetic acid; 2-[4-oxo-5-carboxamido-9-[3-[4-(fluorophenoxy)benzyl]]-9H-pyrido[3,4-b]indolyl]acetic acid; 2-[4-oxo-5-carboxamido-9-[2-fluoro-3-(trifluoromethyl)benzyl]-9H-pyrido[3,4-b]indolyl]acetic acid; 2-[4-oxo-5-carboxamido-9-[2-fluoro-4-(trifluoromethyl)benzyl]-9H-pyrido[3,4-b]indolyl]acetic acid; 2-[4-oxo-5-carboxamido-9-[2-fluoro-5-(trifluoromethyl)benzyl]-9H-pyrido[3,4-b]indolyl]acetic acid; 2-[4-oxo-5-carboxamido-9-[3-fluoro-5-(trifluoromethyl)benzyl]-9H-pyrido[3,4-b]indolyl]acetic acid; 2-[4-oxo-5-carboxamido-9-[4-fluoro-2-(trifluoromethyl)benzyl]-9H-pyrido[3,4-b]indolyl]acetic acid; 2-[4-oxo-5-carboxamido-9-[4-fluoro-3-(trifluoromethyl)benzyl]-9H-pyrido[3,4-b]ndolyl]acetic acid; 2-[4-oxo-5-carboxamido-9-[2-fluoro-6-(trifluoromethyl)benzyl]-9H-pyrido[3,4-b]indolyl]acetic acid; 2-[4-oxo-5-carboxamido-9-(2,3,6-trifluorobenzyl)-9H-pyrido[3,4-b]indolyl]acetic acid; 2-[4-oxo-5-carboxamido-9-(2,3,5-trifluorobenzyl)-9H-pyrido[3,4-b]indolyl]acetic acid; 2-[4-oxo-5-carboxamido-9-(2,4,5-trifluorobenzyl)-9H-pyrido[3,4-b]indolyl]acetic acid; 2-[4-oxo-5-carboxamido-9-(2,4,6-trifluorobenzyl)-9H-pyrido[3,4-b]indolyl]acetic acid; 2-[4-oxo-5-carboxamido-9-(2,3,4-trifluorobenzyl)-9H-pyrido[3,4-b]indolyl]acetic acid; 2-[4-oxo-5-carboxamido-9-(3,4,5-trifluorobenzyl)-9H-pyrido[3,4-b]indolyl]acetic acid; 2-[4-oxo-5-carboxamido-9-[3-(trifluoromethoxyl)benzyl]-9H-pyrido[3,4-b]indolyl]acetic acid; 2-[4-oxo-5-carboxamido-9-[4-(trifluoromethoxyl)benzyl]-9H-pyrido[3,4-b]indolyl]acetic acid; 2-[4-oxo-5-carboxamido-9-[4-methoxy(tetrafluoro)benzyl]-9H-pyrido[3,4-b]indolyl]acetic acid; 2-[4-oxo-5-carboxamido-9-(2-methoxybenzyl)-9H-pyrido[3,4-b]indolyl]acetic acid; 2-[4-oxo-5-carboxamido-9-(3-methoxybenzyl)-9H-pyrido[3,4-b]indolyl]acetic acid; 2-[4-oxo-5-carboxamido-9-(4-methoxybenzyl)-9H-pyrido[3,4-b]indolyl]acetic acid; 2-[4-oxo-5-carboxamido-9-(4-ethylbenzyl)-9H-pyrido[3,4-b]indolyl]acetic acid; 2-[4-oxo-5-carboxamido-9-(4-isopropylbenzyl)-9H-pyrido[3,4-b]indolyl]acetic acid; 2-[4-oxo-5-carboxamido-9-(3,4,5-trimethoxybenzyl)-9H-pyrido[3,4-b]indolyl]acetic acid; 2-[4-oxo-5-carboxamido-9-(3,4-methylenedioxybenzyl)-9H-pyrido[3,4-b]indolyl]acetic acid; 2-[4-oxo-5-carboxamido-9-(4-methoxy-3-methylbenzyl)-9H-pyrido[3,4-b]indolyl]acetic acid; 2-[4-oxo-5-carboxamido-9-(3,5-dimethoxybenzyl)-9H-pyrido[3,4-b]indolyl]acetic acid; 2-[4-oxo-5-carboxamido-9-(2,5-dimethoxybenzyl)-9H-pyrido[3,4-b]indolyl]acetic acid; 2-[4-oxo-5-carboxamido-9-(4-ethoxybenzyl)-9H-pyrido[3,4-b]indolyl]acetic acid; 2-[4-oxo-5-carboxamido-9-(cyclohexylmethyl)-9H-pyrido[3,4-b]indolyl]acetic acid; 2-[4-oxo-5-carboxamido-9-(cyclopentylmethyl)-9H-pyrido[3,4-b]indolyl]acetic acid; 2-[4-oxo-5-carboxamido-9-ethyl-9H-pyrido[3,4-b]indolyl]acetic acid; 2-[4-oxo-5-carboxamido-9-(1-propyl)-9H-pyrido[3,4-b]indolyl]acetic acid; 2-[4-oxo-5-carboxamido-9-(2-propyl)-9H-pyrido[3,4-b]indolyl]acetic acid; 2-[4-oxo-5-carboxamido-9-(1-butyl)-9H-pyrido[3,4-b]indolyl]acetic acid; 2-[4-oxo-5-carboxamido-9-(2-butyl)-9H-pyrido[3,4-b]indolyl]acetic acid; 2-[4-oxo-5-carboxamido-9-isobutyl-9H-pyrido[3,4-b]indolyl]acetic acid; 2-[4-oxo-5-carboxamido-9-[2-(1-phenylethyl)]-9H-pyrido[3,4-b]indolyl]acetic acid; 2-[4-oxo-5-carboxamido-9-[3-(1-phenylpropyl)]-9H-pyrido[3,4-b]indolyl]acetic acid; 2-[4-oxo-5-carboxamido-9-[4-(1-phenylbutyl)]-9H-pyrido[3,4-b]indolyl]acetic acid; 2-[4-oxo-5-carboxamido-9-(1-pentyl)-9H-pyrido[3,4-b]indolyl]acetic acid; 2-[4-oxo-5-carboxamido-9-(1-hexyl)-9H-pyrido[3,4-b]indolyl]acetic acid; 4-[(9-benzyl-4-carbamoyl-1,2,3,4-tetrahydrocarbazol-6-yl)oxy]butyric acid; 3-[(9-benzyl-4-carbamoyl-1,2,3,4-tetrahydrocarbazol-6-yl)oxy]propylphosphonic acid; 2-[(9-benzyl-4-carbamoyl-1,2,3,4-tetrahydrocarbazol-6-yl)oxy]methylbenzoic acid; 3-[(9-benzyl-4-carbamoyl-7-n-octyl-1,2,3,4-tetrahydrocarbazol-6-yl)oxy]propylphosphonic acid; 4-[(9-benzyl-4-carbamoyl-7-ethyl-1,2,3,4-tetrahydrocarbazol-6-yl)oxy]butyric acid; 3-[(9-benzyl-4-carbamoyl-7-ethyl-1,2,3,4-tetrahydrocarbazol-6-yl)oxy]propylphosphonic acid; 3-[(9-benzyl-4-carbamoyl-7-ethyl-1,2,3,4-tetrahydrocarbazol-6-yl)oxy]propylphosphonic acid; (S)-(+)-4-[(9-benzyl-4-carbamoyl-7-ethyl-1,2,3,4-tetrahydrocarbazol-6-yl)oxy]butyric acid; 4-[9-benzyl-4-carbamoyl-6-(2-cyanoethyl)-1,2,3,4-tetrahydrocarbazol-6-yl]oxybutyric acid; 4-[9-benzyl-4-carboxamido-7-(2-phenylethyl)-1,2,3,4-tetrahydrocarbazol-6-yl]oxybutyric acid; 4-[9-benzyl-4-carboxamidocarbazol-6-yl]oxybutyric acid; methyl 2-[(9-benzyl-4-carbamoyl-1,2,3,4-tetrahydrocarbazol-6-yl)oxy]methylbenzoate; 4-[9-benzyl-4-carbamoyl-7-(2-cyanoethyl)-1,2,3,4-tetrahydrocarbazol-6-yl]oxybutyric acid; 9-benzyl-7-methoxy-5-cyanomethyloxy-1,2,3,4-tetrahydrocarbazole-4-carboxamide; [9-benzyl-4-carbamoyl-8-methyl-carbazole-5-yl]oxyacetic acid; and [9-benzyl-4-carbamoyl-carbazole-5-yl]oxyacetic acid, or pharmaceutically acceptable salts, solvates, prodrug derivatives, racemates, tautomers, or optical isomers thereof.

Certain embodiments of the methods and compositions provided herein utilize the sPLA₂ inhibitor 3-(2-Amino-1,2-dioxoethyl)-2-ethyl-1-(phenylmethyl)-1H-indol-4-yl)oxy)acetic acid (A-001, also referred to in the art as S-5920 or LY315920) or a salt, solvate, or prodrug thereof. Certain embodiments utilize the sodium salt of A-001. A-001 has the structure:

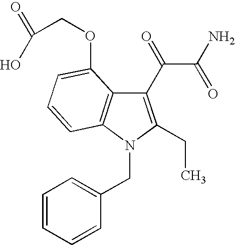

A-001 is a competitive inhibitor of sPLA₂.

Certain embodiments of the methods and compositions provided herein utilize an A-001 prodrug, and in certain of these embodiments the prodrug is a C₁-C₆ alkyl ester, acyloxyalkyl ester, or alkyloxycarbonyloxyalkyl ester of A-001. In certain of these embodiments, the prodrug is A-002 (also referred to in the art as S-3013, LY333013, or varespladib methyl), which has the structure:

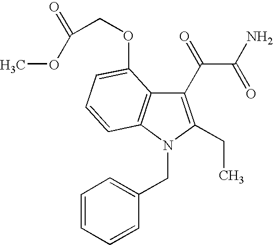

A-002, which has a terminal half-life (t_{1/2}) of approximately ten hours, is rapidly absorbed and hydrolyzed to the active A-001 molecule. One skilled in the art will recognize that other prodrug forms of A-001 may be used in the methods and compositions disclosed herein. One skilled in the art would recognize that any prodrug that is metabolized to the active A-001 molecule would be likely to have similar therapeutic characteristics, and such a skilled artisan could identify such prodrugs with minimal experimentation.

In those embodiments of the compositions and methods disclosed herein that utilize statins, examples of statins that may be used include, but are not limited to, atorvastatin or atorvastatin calcium (marketed as Lipitor® or Torvast®; see, e.g., U.S. Pat. Nos. 4,681,893 or 5,273,995) and atorvastatin combinations (e.g., atorvastatin plus amlodipine (marketed as Norvasc®), combination marketed as Caduet®, see, e.g., U.S. Pat. No. 6,455,574; atorvastatin plus CP-529414 (marketed as Torcetrapib®); atorvastatin plus APA-01; atorvastatin plus ezetimibe), cerivastatin (marketed as Lipobay® or Baycol®), fluvastatin (marketed as Lescol®; U.S. Pat. No. 4,739,073), lovastatin (marketed as Mevacor® or Altocor®; see, e.g., U.S. Pat. No. 4,231,938), lovastatin combinations (e.g., lovastatin plus Niaspan®, combination marketed as Advicor®), mevastatin, pitavastatin (marketed as Livalo® or Pitava®), pravastatin (marketed as Pravachol®, Mevalotin®, Selektine®, or Lipostat®; see, e.g., U.S. Pat. No. 4,346,227), pravastatin combinations (e.g., pravastatin plus fenofibrate), rosuvastatin (marketed as Crestor®), rosuvastatin combinations (e.g., rosuvastatin plus TriCor®), simvastatin (marketed as Zocor® or Lipex®; see, e.g., U.S. Pat. Nos. 4,444,784; 4,916,239; and 4,820,850), and simvastatin combinations (e.g., simvastatin plus ezetimibe, combination marketed as Vytorin®, see, e.g., U.S. Pat. No. 7,229,982; simvastatin plus Niaspan®, combination marketed as Simcor®; simvastatin plus MK-0524A, combination referred to as MK-0524B), as well as various pharmaceutically acceptable salts, solvates, salts, stereoisomers, prodrugs derivatives, or nitroderivatives of the compounds listed above. In some cases, such as for example with simvastatin, the active form of the statin is a metabolite formed in the body of a subject following administration. In other cases, statins are administered in their active form. In certain embodiments, statins may be administered according to their standard recommended dosage, while in other embodiments statins may be administered lower than the recommended dosage.

In certain embodiments, methods are provided for inhibiting inflammation in a subject in need thereof by administering a therapeutically effective amount of one or more sPLA₂ inhibitors alone or in combination with one or more statins. In certain embodiments, the subject has previously been diagnosed with ACS. In certain embodiments, the subject is classified as unstable, and in certain of these embodiments the subject has previously experienced an ACS event and/or been diagnosed with one or more symptoms associated with an ACS event. In certain of these embodiments, the occurrence of the ACS event was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has experienced an ACS event within 96 hours of the first administration of the one or more sPLA₂ inhibitors. In other embodiments, the diagnosis of the ACS event or associated symptom was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has been diagnosed with an ACS event within 96 hours of the first administration of the one or more sPLA₂ inhibitors. In certain embodiments, administration of one or more sPLA₂ inhibitors alone or in combination with one or more statins results in a decrease in blood, serum, and/or plasma levels of one or more inflammatory markers such as hs-CRP, sPLA₂, and/or IL-6. In certain embodiments, the decrease in inflammatory marker levels is first observed within 1-6 days, 1-2 weeks, 2-4 weeks, or 4-6 weeks after the first administration of one or more sPLA₂ inhibitors. In certain embodiments, the decrease in inflammatory marker levels is also observed at later timepoints, such as within 6-8 weeks, 8-10 weeks, 10-12 weeks, 12-14 weeks, or 14-16 weeks after the first administration of one or more sPLA₂ inhibitors. In certain embodiments, inhibition of inflammation results in prevention and/or reduction of inflammation. In certain embodiments wherein one or more sPLA₂ inhibitors are administered in conjunction with one or more statins, the resultant decrease in inflammation and/or inflammatory marker levels is greater than the decrease obtained by administering one or more statins alone. In certain embodiments, sPLA₂ inhibitors and/or statins are administered in conjunction with one or more pharmaceutically acceptable carriers. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin.

In certain embodiments, methods are provided for treating dyslipidemia in a subject in need thereof by administering a therapeutically effective amount of one or more sPLA₂ inhibitors alone or in combination with one or more statins. In certain embodiments, the subject has previously been diagnosed with ACS. In certain embodiments, the subject is classified as unstable, and in certain of these embodiments the subject has previously experienced an ACS event and/or been diagnosed with one or more symptoms associated with an ACS event. In certain embodiments, the occurrence of the ACS event was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has experienced an ACS event within 96 hours of the first administration of the one or more sPLA₂ inhibitors. In other embodiments, the diagnosis of the ACS event or associated symptom was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has been diagnosed with an ACS event within 96 hours of the first administration of the one or more sPLA₂ inhibitors. In certain embodiments, administration of one or more sPLA₂ inhibitors alone or in combination with one or more statins results in a decrease in blood, serum, and/or plasma cholesterol levels, such as for example LDL-C, non-HDL cholesterol, and/or total cholesterol. In certain of these embodiments, administration of one or more sPLA₂ inhibitors starting within 96 hours of an ACS event results in a decrease in LDL-C levels. In certain embodiments, the decrease in cholesterol levels such as LDL-C levels is first observed within 1-6 days, 1-2 weeks, 2-4 weeks, or 4-6 weeks after the first administration of one or more sPLA₂ inhibitors. As discussed above, LDL levels generally decrease slightly immediately following an ACS event. In certain embodiments, administration of one or more sPLA₂ inhibitors decreases cholesterol levels more rapidly and/or to a greater degree than is normally observed during this period of natural LDL reduction. In those embodiments wherein one or more sPLA₂ inhibitors are administered in conjunction with one or more statins, the decrease in cholesterol levels during this time period may be greater than the decrease obtained by administration of one or more statins alone. In certain embodiments, the decrease in cholesterol levels is also observed at later timepoints, such as within 6-8 weeks, 8-10 weeks, 10-12 weeks, 12-14 weeks, or 14-16 weeks after the first administration of one or more sPLA₂ inhibitors. In certain of these embodiments, administration of one or more sPLA₂ inhibitors prevents, reduces, and/or slows the natural increase in LDL levels that normally follows the initial post-ACS event LDL drop. In certain embodiments wherein one or more sPLA₂ inhibitors are administered in conjunction with one or more statins, the resultant decrease in cholesterol levels during this time period is greater than the decrease obtained by administering one or more statins alone. In certain embodiments, cholesterol levels are decreased to a specific target level at one or more timepoints after the first administration of one or more sPLA₂ inhibitors. For example, administration of one or more sPLA₂ inhibitors may decrease LDL-C levels to a specific target level, such as for example to 100 mg/dl or less, 90 mg/dl or less, 80 mg/dl or less, 70 mg/dl or less, 60 mg/dl or less, or 50 mg/dl or less at various timepoints after the first administration, such as for example at 1 week, 2 weeks, 4 weeks, 8 weeks, or 16 weeks. In certain of these embodiments, LDL-C levels are decreased to 70 mg/dl or less, which corresponds to the Adult Treatment Program III (ATP III) target level for LDL-C. In certain embodiments, sPLA₂ inhibitors and/or statins are administered in conjunction with one or more pharmaceutically acceptable carriers. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin.

In certain embodiments, methods are provided for reducing cholesterol and/or inflammatory marker levels to a pre-determined target level by administering a therapeutically effective amount of one or more sPLA₂ inhibitors alone or in combination with one or more statins. In certain embodiments, the subject has previously been diagnosed with ACS. In certain embodiments, the subject is classified as unstable, and in certain of these embodiments the subject has previously experienced an ACS event and/or been diagnosed with one or more symptoms associated with an ACS event. In certain embodiments, the occurrence of the ACS event was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has experienced an ACS event within 96 hours of the first administration of the one or more sPLA₂ inhibitors. In other embodiments, the diagnosis of the ACS event or associated symptom was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has been diagnosed with an ACS event within 96 hours of the first administration of the one or more sPLA₂ inhibitors. In certain embodiments, LDL-C levels are decreased to a target level of 100 mg/dl or less, 90 mg/dl or less, 80 mg/dl or less, 70 mg/dl or less, 60 mg/dl or less, or 50 mg/dl or less. In certain of these embodiments, LDL-C levels are decreased to a target level of 70 mg/dl or less. In certain embodiments, hs-CRP levels are decreased to a target level of 5 mg/L or less, 3 mg/L or less, or 1 mg/L or less. In certain of these embodiments, hs-CRP levels are decreased to a target level of 3 mg/L or less. In certain embodiments, a single biomarker target level is reached, while in other embodiments target levels may be set and achieved for multiple biomarkers. For example, administration of one or more sPLA₂ inhibitors alone or in combination with statin may be used to reach a target level for LDL-C, hs-CRP, sPLA₂, IL-6, or a combination thereof. In certain embodiments, administration of one or more sPLA₂ inhibitors decreases cholesterol and/or inflammatory marker levels to a pre-determined target level within a specific time period, such as for example within 1-6 days, 1-2 weeks, 2-4 weeks, or 4-6 weeks after the first administration of one or more sPLA₂ inhibitors. In certain of these embodiments, administration of one or more sPLA₂ inhibitors keeps cholesterol and/or inflammatory marker levels at or below the target level for some period of time after the target level is initially reached, such as for example out to 6-8 weeks, 8-10 weeks, 10-12 weeks, 12-14 weeks, or 14-16 weeks after the first sPLA₂ inhibitor administration. In certain embodiments, administration of one or more sPLA₂ inhibitors in conjunction with one or more statins reduces cholesterol and/or inflammatory marker levels to a pre-determined target level more quickly than administration of one or more statin only. Alternatively or in addition to this effect, administration of one or more sPLA₂ inhibitors in conjunction with one or more statins may keep cholesterol and/or inflammatory marker levels at or below the pre-determined target level for a longer time period after lowering levels to the target level. In certain embodiments, administration of one or more sPLA₂ inhibitors and/or one or more statins is discontinued when the subject reaches a specific target level. In other embodiments, administration continues after the target level is reached. In certain embodiments, sPLA₂ inhibitors and/or statins are administered in conjunction with one or more pharmaceutically acceptable carriers. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin.

In certain embodiments of the methods disclosed herein, administration of one or more sPLA₂ inhibitors alone or in combination with one or more statins may result in a decrease in inflammation, inflammatory markers (including hs-CRP, sPLA₂, and/or IL-6), and/or cholesterol levels (including LDL-C, non-HDL cholesterol, and/or total cholesterol) over the entire course of drug administration, meaning that subjects receiving sPLA₂ inhibitor or sPLA₂ inhibitor/statin treatment exhibit lower levels of inflammation, inflammatory markers, and/or cholesterol than subjects receiving no treatment or treatment with statin alone at all or most timepoints following the first administration of sPLA₂ inhibitor. In other embodiments, administration of one or more sPLA₂ inhibitors alone or in combination one or more statins reduces inflammation and/or cholesterol levels to a greater extent than no treatment or treatment with statins alone at the early stages of drug administration, with the A-002 and A-002/statin subjects eventually exhibiting the same or nearly the same levels of inflammation or cholesterol as control or statin only subjects. For example, subjects administered A-002 plus statin may exhibit greater decreases in hs-CRP, sPLA₂, IL-6, and/or LDL levels than subjects administered statins only immediately or soon after the first administration of A-002, with the relative difference in hs-CRP, sPLA₂, IL-6, and/or LDL-C reduction eventually leveling out at later timepoints. In these embodiments, A-002 plus statin may lower hs-CRP, sPLA₂, IL-6, and/or LDL-C levels more effectively than statin alone at one or more timepoints from 0 to 28 weeks after the first A-002 administration, such as for example at 1 hour, 12 hours, 24 hours, 2 days, 1 week, 2 weeks, 4 weeks, 6 weeks, 8 weeks, 10 weeks, 12 weeks, 14 weeks, 16 weeks, 20 weeks, 24 weeks, or 28 weeks after the first A-002 administration. Since subjects experiencing an ACS event exhibit a marked increase in inflammation (and inflammatory marker levels) immediately following the ACS event and this increase is associated with increase MACE occurrence, the ability of A-002 alone or in combination with statin to reduce inflammation more quickly than statin alone is particularly relevant to treatment of MACEs.

In certain embodiments, methods are provided for treating MACEs in a subject in need thereof by administering a therapeutically effective amount of one or more sPLA₂ inhibitors alone or in combination with one or more statins. In certain embodiments, the subject has previously been diagnosed with ACS. In certain embodiments, the subject is classified as unstable, and in certain of these embodiments the subject has previously experienced an ACS event and/or been diagnosed with one or more symptoms associated with an ACS event. In certain embodiments, the occurrence of the ACS event was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has experienced an ACS event within 96 hours of the first administration of the one or more sPLA₂ inhibitors. In other embodiments, the diagnosis of the ACS event or associated symptom was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has been diagnosed with an ACS event within 96 hours of the first administration of the one or more sPLA₂ inhibitors. In certain embodiments, sPLA₂ inhibitors and/or statins are administered in conjunction with one or more pharmaceutically acceptable carriers. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin.

In certain embodiments, administration of one or more sPLA₂ inhibitors alone or in combination with one or more statins is more effective at treating MACEs than administration of one or more statins alone over a particular time period. For example, administration of one or more sPLA₂ inhibitors alone or in combination with one or more statins may be more effective than administration of one or more statins at treating MACEs over a period of 2 weeks, 4 weeks, 6 weeks, 8 weeks, 10 weeks, 12 weeks, 14 weeks, 16 weeks, 20 weeks, 24 weeks, or 28 weeks after the subject experienced an ACS event, was diagnosed as having experienced an ACS event, and/or received the first administration of sPLA₂ inhibitor. This increased effectiveness may result in prevention of MACE occurrence, a decrease in the likelihood of MACE occurrence, a decrease in the severity of MACE occurrence, and/or a delay in MACE occurrence. In certain embodiments, the improvement in MACE treatment is observed across the entire spectrum of MACEs or across a defined set of MACEs. In other embodiments, the improvement in MACE treatment may be observed only in one or more specific types of MACE (e.g., cardiovascular death, fatal or non-fatal MI, UA (including UA requiring urgent hospitalization), fatal or non-fatal stroke, and/or need for revascularization procedures). In certain embodiments, administration of one or more sPLA₂ inhibitors alone or in combination with one or more statins may shift the likelihood of MACE occurrence from more severe to less severe forms. For example, administration of sPLA₂ inhibitor alone or in combination with one or more statins may reduce the number of fatal MACEs versus administration of statin alone, but have no effect on the overall number of MACEs.

In certain embodiments, methods are provided for treating ACS in a subject in need thereof by administering a therapeutically effective amount of one or more sPLA₂ inhibitors alone or in combination with one or more statins. In certain embodiments, the subject has previously been diagnosed with ACS. In certain embodiments, the subject is classified as unstable, and in certain of these embodiments the subject has previously experienced an ACS event and/or been diagnosed with one or more symptoms associated with an ACS event. In certain embodiments, the occurrence of the ACS event was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has experienced an ACS event within 96 hours of the first administration of the one or more sPLA₂ inhibitors. In other embodiments, the diagnosis of the ACS event or associated symptom was recent, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has been diagnosed with an ACS event within 96 hours of the first administration of the one or more sPLA₂ inhibitors. In certain embodiments, sPLA₂ inhibitors and/or statins are administered in conjunction with one or more pharmaceutically acceptable carriers. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin. In certain embodiments, administration of one or more sPLA₂ inhibitors alone or in combination with one or more statins is more effective at treating ACS than administration of one or more statins alone. In certain embodiments, this increased effectiveness may result in a reduction in ACS event occurrence, a decrease in the likelihood of ACS event occurrence, a decrease in the severity of ACS event occurrence, and/or a delay in ACS event occurrence. In certain embodiments, the improvement in ACS treatment is observed across the entire spectrum of conditions associated with ACS. In other embodiments, the improvement in ACS treatment may be observed only in one or more specific conditions associated with ACS (e.g., UA, STEMI, and/or NSTEMI).

In certain embodiments, the use of one or more sPLA₂ inhibitors as an adjunct therapy to statin following an ACS event to reduce the risk of MACEs is provided. In certain of these embodiments, the one or more sPLA₂ inhibitors include A-001 or a prodrug thereof, and in certain of these embodiments the prodrug thereof is A-002. In certain embodiments, the statin is atorvastatin, rosuvastatin, and/or simvastatin. In certain embodiments, administration of one or more sPLA₂ inhibitors as an adjunct therapy to statins following an ACS event reduces the risk of one or more MACEs including cardiovascular death, fatal or non-fatal MI, UA including UA requiring urgent hospitalization, fatal or non-fatal stroke, and revascularization procedures. In certain embodiments, the first administration of sPLA₂ inhibitor takes place within 24 hours, 24 to 48 hours, 48 to 96 hours, 96 hours to 1 week, 1 to 2 weeks, 2 to 6 weeks, or 6 to 12 weeks of the occurrence or diagnosis of an ACS event. In certain of these embodiments, the first administration of sPLA₂ inhibitor takes place within 96 hours of the occurrence or diagnosis of the ACS event. In certain embodiments, the use of A-002 in combination with any dosage of statin is provided, wherein A-002 is first administered within 96 hours of an ACS event and is administered for up to 16 weeks, and wherein administration results in prevention of cardiovascular death, non-fatal MI, non-fatal stroke, or UA requiring urgent hospitalization. In other embodiments, the use of A-002 in combination with any dosage of atorvastatin or rosuvastatin is provided, wherein A-002 is first administered within 96 hours of an ACS event and is administered for up to 90 days, and wherein administration results in prevention of all-cause mortality, non-fatal MI, non-fatal stroke, or UA requiring urgent hospitalization.

In certain embodiments, methods are provided for increasing the effectiveness of one or more therapeutics used in the treatment of CVD, MACEs, or ACS by administering one or more sPLA₂ inhibitors. In certain embodiments, the one or more sPLA₂ inhibitors include A-001 or a salt, solvate, or prodrug thereof, and in certain of these embodiments the prodrug thereof is A-002. In certain embodiments, the other therapeutics used in the treatment of CVD, MACEs, or ACS are statins, aspirin, ACE inhibitors, beta-blockers, anti-platelet therapeutics, and/or anti-coagulant therapeutics. An increase in effectiveness of another therapeutic used in the treatment of CVD, MACEs, or ACS as used herein refers to an increase the treatment effect of the therapeutic, a decrease in the dosage of the therapeutic required to obtain a particular level of treatment effect, or some combination thereof.

In certain embodiments of the methods provided herein, one or more additional therapeutics used in the treatment of CVD, MACEs, or ACS may be administered to a subject in conjunction with one or more sPLA₂ inhibitors or one or more sPLA₂ inhibitors and one or more statins. For example, sPLA₂ inhibitors and statins may be administered in conjunction with one or more of aspirin, ACE inhibitors, beta-adrenergic blockers, and/or anti-platelet therapy.

As disclosed herein, A-002 in combination with statin significantly decreased inflammatory marker levels in subjects with diabetes who had recently experienced an ACS event. This establishes that A-002 plus statin has anti-inflammatory effects in post-ACS subjects that were previously diagnosed with an inflammatory condition. Therefore, in certain embodiments of the methods disclosed herein, the subject being treated has been diagnosed with or exhibited one or more symptoms of a condition associated with inflammation or high inflammatory marker levels, such as for example diabetes, metabolic syndrome, arthritides, vasculitides, chronic kidney disease, obesity, autoimmune diseases such as psoriasis, chronic obstructive pulmonary disorder (COPD), or infection. In certain embodiments, the subject may have been diagnosed with or exhibited symptoms of one or more of these conditions prior to occurrence of an ACS event. In other embodiments, the first diagnosis or symptom onset may occur after an ACS event. In certain embodiments, the subject being treated may be a smoker.

In certain embodiments of the methods provided herein, the one or more sPLA₂ inhibitors may be administered via different routes and/or in different forms at different times over the course of treatment. For example, in certain embodiments the one or more sPLA₂ inhibitors may be administered via a parenteral route such as infusion in the hours and days immediately following the ACS event, followed by administration via a different route at later timepoints. These embodiments allow for rapid administration of sPLA₂ inhibitor in the hours and/or days immediately following an ACS event. In addition, they allow for easier administration of the compound to a subject who is incapacitated or partially incapacitated. The form of the drug may vary depending on the administration route being used. For example, in certain embodiments A-001 may be administered via a parenteral route in the early timepoints after an ACS event. At later timepoints, parenteral administration may be phased out and replaced with oral administration of A-002 or another prodrug form of A-001. The phase out from parenteral to oral administration may occur gradually, with parenteral administration being reduced over a series of timepoints while oral administration is simultaneously increased. Alternatively, parenteral administration may be discontinued all at once, and the subject may be switched immediately to a full oral dosage of the drug. In other embodiments, a subject may receive sPLA₂ inhibitors in different forms and/or via different administration routes throughout the entire course of treatment. Administering sPLA₂ inhibitors via a parenteral route in the time period immediately after an ACS event may be advantageous in certain embodiments because it allows for therapeutic blood levels of the drug to be obtained rapidly more. In addition, it allows for blood levels of the drug to be maintained at more steady levels.

In certain embodiments of the methods provided herein wherein one or more sPLA₂ inhibitors and one or more statins are administered to a subject, the one or more sPLA₂ inhibitors and one or more statins may be administered separately, i.e., in separate compositions. In these embodiments, the one or more sPLA₂ inhibitors and one or more statins may be administered simultaneously or sequentially. Further, one or more sPLA₂ inhibitors and one or more statins may be administered at different times, and one compound may be administered more frequently than another. In certain embodiments wherein one or more sPLA₂ inhibitors and one or more statins are given in multiple administrations, one or both may be administered anywhere from one or more times per day to once every week, once every month, or once every several months. In certain of these embodiments, the one or more sPLA₂ inhibitors and/or one or more statins may be administered once a day, twice a day, or three times a day. Alternatively, the one or more sPLA₂ inhibitors and one or more statins may be administered continuously or semi-continuously, such as for example by intravenous infusion. In certain embodiments, administration of one or more sPLA₂ inhibitors and one or more statins may begin at the same time. In these embodiments, administration of sPLA₂ inhibitor and statin may begin within a certain time period after an ACS event or diagnosis of an ACS event, such as for example within 96 hours. In other embodiments, administration of one or more sPLA₂ inhibitors and one or more statins may begin at different times. In these embodiments, either compound may be administered first. For example, one or more sPLA₂ inhibitors may be administered first within a certain time period after an ACS event or diagnosis of an ACS event, such as for example within 96 hours of the event, with administration of statin beginning at a later timepoint. Alternatively, administration of one or more statins may begin before administration of one or more sPLA₂ inhibitors. In these embodiments, the subject may already have been on statin prior to the ACS event. When the subject was already on statin prior to the ACS event, statin administration after the event may continue at the same dosage and administration interval as before the ACS event. Alternatively, the dosage and/or administration interval of the statin may be adjusted after the ACS event. In addition, the specific statin being administered may be changed following the ACS event. For example, a subject that was receiving rosuvastatin prior to an ACS event may switch to atorvastatin following the event, or vice versa.

In other embodiments, one or more sPLA₂ inhibitors and one or more statins may be administered as part of a single composition. Provided herein in certain embodiments are such compositions, as well as kits comprising these compositions and the use of one or more sPLA₂ inhibitors and one or more statins in producing these compositions. In those embodiments wherein one or more sPLA₂ inhibitors and one or more statins are administered to a subject as a single composition, the composition may be administered on a one-time basis or in multiple administrations. In those embodiments wherein the composition is given in multiple administrations, it may be administered anywhere from one or more times per day to once every week, once every month, or once every several months. In certain of these embodiments, the composition may be administered once a day, twice a day, or three times a day. Alternatively, the composition may be administered continuously or semi-continuously, such as for example by parenteral administration. In certain embodiments, the composition may comprise one or more additional CVD therapeutics, such as for example aspirin, ACE inhibitors, beta-adrenergic blockers, and/or anti-platelet therapy.

One or more sPLA₂ inhibitors, one or more statins, or compositions comprising one or more sPLA₂ inhibitors and one or more statins may be administered on a one-time basis, continuously, or at set intervals over a particular time period. In those embodiments wherein the compounds are administered over a particular time period, the time period may be determined in advance and may be measured in weeks or days. For example, in certain embodiments one or more sPLA₂ inhibitors may be administered at set intervals over a 2 week, 4 week, 6 week, 8 week, 10 week, 12 week, 14 week, 15 week, 16 week, 17 week, 18 week, 19 week, 20 week, 24 week, or 28 week period. In certain of these embodiments, one or more sPLA₂ inhibitors are administered for up to 16 weeks. In certain embodiments, one or more sPLA₂ inhibitors may be administered for up to 70 days, up to 80 days, up to 90 days, up to 100 days, up to 110 days, up to 112 days, up to 115 days, or up to 120 days. In certain of these embodiments, one or more sPLA₂ inhibitors are administered for up to 112 days. Alternatively, the duration of administration may be based on reaching a particular therapeutic benchmark. For example, in certain embodiments, one or more sPLA₂ inhibitors may be administered at set intervals until inflammation decreases to a specified degree. In certain embodiments, the specified decrease in inflammation may be measured by the level of one or more inflammatory markers such as hs-CRP, sPLA₂, and/or IL-6. For example, the specified decrease in inflammation may occur when hs-CRP, sPLA₂, and/or IL-6 levels drop by 20%, 40%, 60%, or 80% from levels observed just prior to the first sPLA₂ inhibitor administration. In other embodiments, one or more sPLA₂ inhibitors may be administered at set intervals until cholesterol levels decrease to a specified degree. Alternatively or in addition to these embodiments, one or more sPLA₂ inhibitors may be administered until one or more symptoms associated with ACS or MACE risk decreases or disappears.

In certain embodiments, methods are provided for preventing cardiovascular death, non-fatal MI, non-fatal stroke, and/or UA requiring urgent hospitalization in a subject who has experienced an ACS event within the past 96 hours by administering A-001 or a salt, solvate, or prodrug thereof and any statin at regular intervals for a maximum of 16 weeks. In certain of these embodiments the prodrug thereof is A-002. In certain embodiments, the interval at which A-001 or a salt, solvate, or prodrug thereof is administered is once, twice, or three times daily. In certain embodiments, A-001 or a salt, solvate, or prodrug thereof is administered continuously or semi-continuously.

Compositions comprising one or more sPLA₂ inhibitors and/or one or more statins may be administered by any administration pathway known in the art, including but not limited to oral, aerosol, enteral, nasal, ophthalmic, parenteral, or transdermal (e.g., topical cream or ointment, patch). “Parenteral” refers to a route of administration that is generally associated with injection, such as for example bolus injection or continuous or semi-continuous infusion. Parenteral administration may be accomplished by a variety of pathways, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. One or more sPLA₂ inhibitors, one or more statins, or combined sPLA₂ inhibitor/statin compositions as described herein may be administered in any pharmaceutically acceptable form, including for example in the form of a solid, liquid solution, suspension, emulsion, dispersion, micelle, or liposome. Preparations for injection may include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile emulsions. The solutions may be either aqueous or nonaqueous. In certain embodiments, the compositions may comprise one or more pharmaceutically acceptable carriers or may be administered in conjunction with one or more pharmaceutically acceptable carriers.

In certain embodiments, pharmaceutical compositions comprising one or more sPLA₂ inhibitors or one or more sPLA₂ inhibitors and one or more statins may be formed into oral dosage units, such as for example tablets, pills, or capsules. Such an oral dosage unit may comprise the active ingredients (e.g., A-002 and atorvastatin) and one or more pharmaceutically acceptable carriers. In certain embodiments, pharmaceutical compositions comprising one or more sPLA₂ inhibitors and/or one or more statins may be administered via a time release delivery vehicle, such as for example a time release oral dosage unit. A “time release vehicle” as used herein refers to any delivery vehicle that releases active agent (e.g., A-002 and atorvastatin) at some time after administration or over a period of time following administration rather than immediately upon administration. Time release may be obtained by a coating on the vehicle that dissolves over a set timeframe following administration. In certain embodiments, the time release vehicle may comprise multiple layers of coating alternated with multiple layers of active ingredients, such that each layer of coating releases a certain volume of active ingredients as it dissolves. In other embodiments, one or more sPLA₂ inhibitors and/or one or more statins may be administered via an immediate release delivery vehicle.

A therapeutically effective amount of one or more sPLA₂ inhibitors and/or one or more statins may be determined for each compound individually. For example, statins may be administered or included in a pharmaceutical composition at a dosage that is well known in the art to decrease cholesterol levels. In these embodiments, statins may be administered according to the manufacturer instructions for the particular statin. In certain of these embodiments, a particular statin may be administered at a dosage ranging from about 5 mg to about 80 mg. For example, in those embodiments wherein the statin is atorvastatin, simvastatin, or rosuvastatin, the statin may be administered at a dosage of about 5, 10, 20, 40, 60, or 80 mg. One skilled in the art will recognize that in those embodiments wherein one or more statins are combined with one or more sPLA₂ inhibitors in a single composition, the amount of statin that constitutes a therapeutically effective amount may be different than the amount of statin that constitutes a therapeutically effective amount when administered alone due to, for example, interactions between the statin and the sPLA₂ inhibitor. For example, the effective dosage of a statin for use in combination therapy may be lower than the effective dosage for the statin when administered alone. Likewise, the therapeutically effective amount of an sPLA₂ inhibitor may be lower when administered in conjunction with a statin than when the sPLA₂ inhibitor is administered alone. In these situations, one skilled in the art will readily be able to determine a therapeutically effective amount for the combination using methods well known in the art. In certain embodiments, a therapeutically effective amount of one or more sPLA₂ inhibitors for use either alone or in combination with one or more statins is about 25 to about 5,000 mg/dose, and in certain of these embodiments a therapeutically effective amount may be from about 50 to about 1,000 mg/dose. The therapeutically effective amount of an sPLA₂ inhibitor or statin may change over the course of administration. For example, dosages may be increased or decreased as necessary in the weeks following an ACS event based on therapeutic response, side effects, and/or other factors.

In certain embodiments, kits are provided for reducing inflammation and/or inflammatory marker levels, treating dyslipidemia (e.g., lowering total cholesterol or LDL-C), and/or treating MACEs or ACS comprising one or more sPLA₂ inhibitors. In certain embodiments, these kits further comprise one or more statins. In certain embodiments, the one or more sPLA₂ inhibitors include A-001 or a salt, solvate, or prodrug thereof, and in certain of these embodiments the prodrug thereof is A-002. In certain embodiments, the kits provided herein further comprise instructions for usage, such as dosage or administration instructions.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### Examples

### Example 1

### Effect of A-002 Plus Statin on Major Adverse Cardiac Events and Serum Lipid Levels in Human ACS Subjects

625 adult (18 years of age or older) unstable human subjects who had recently experienced an index ACS event (UA, NSTEMI, or STEMI) were randomized to receive placebo or A-002 at 500 mg once daily via oral administration in a double-blinded manner. A-002 was delivered in the form of two 250 mg tablets. In addition, all subjects received atorvastatin at 80 mg once daily via oral administration of a single tablet. Subtypes of index ACS events were similarly distributed between the A-002/atorvastatin group and the atorvastatin only group.

Subjects were randomized within 96 hours of hospital admission for an index ACS event, or within 96 hours of index ACS event diagnosis if already hospitalized. Prior to randomization, subjects were screened for pertinent medical history, and baseline levels of LDL and hs-CRP were measured. Baseline sPLA₂ levels were also measured in a random subset of subjects. Percutaneous revascularization, if required or planned for a particular subject, was performed prior to randomization.

In addition to an index ACS event, all subjects exhibited one or more of the following: diabetes; a BMI of 25 kg/m² or greater; serum hs-CRP levels of 2 mg/L or greater if diagnosed with NSTEMI or STEMI or 3 mg/L or greater if diagnosed with UA; or at least three characteristics of metabolic syndrome (waist circumference greater than 102 cm (male) or 88 cm (female), serum TG levels of 150 mg/dL (1.7 mmol/L) or greater, HDL levels less than 40 mg/dL (1.0 mmol/L) (male) or 50 mg/dL (1.3 mmol/L) (female), blood pressure of 130/85 mm Hg or greater, or plasma glucose of 110 mg/dL (6.1 mmol/L) or greater. Subjects were excluded if they were receiving statin therapy at maximum recommended or tolerated dosage (i.e., 40-80 mg QD for atorvastatin, fluvastatin, lovastatin, pravastatin, or simvastatin, or 20-40 mg QD rosuvastatin) at the time of the index ACS event. During the trial, subjects were prevented from using any lipid-lowering therapy other than 80 mg atorvastatin and/or A-002.

For purposes of this study, subjects were defined as having UA if they exhibited: 1) chest pain or angina occurring at rest or with minimal exertion, lasting longer than ten minutes, and consistent with myocardial ischemia within 24 hours prior to hospitalization; 2) an ECG reading with new or dynamic ST or T wave changes of 1 mm or greater, horizontal or down sloping ST segment depression not previously present in at least two contiguous leads, or new wall motion or reversible perfusion abnormalities; and 3) cardiac troponin I levels of 0.1 ng/ml or greater but less than upper limit of normal (ULN) or cardiac troponin T levels of 0.2 ng/ml or greater.

Subjects were defined as having NSTEMI if they exhibited: 1) no ECG changes, ST depression, or T wave changes (i.e., no new Q waves on serial ECGs) and 2) an increase in cardiac troponin greater than the local limit for the definition of MI or an increase in CK-MB isoenzyme greater than ULN.

Subject were defined as having STEMI if they exhibited: 1) persistent ST or T wave changes or ST segment elevation of at least 2 mm in two contiguous leads and persisting longer than 15 minutes, and 2) an increase in cardiac troponin greater than the local limit for the definition of MI or an increase in CK-MB greater than ULN.

Individual subjects received treatment until all subjects had been treated for a minimum of 24 weeks or until the occurrence of a MACE. For purposes of this study, MACEs included all-cause mortality, non-fatal MI, documented UA requiring urgent hospitalization, revascularization occurring 60 days or more after the initial index ACS event, and non-fatal stroke. Subjects were evaluated at 2, 4, 8, 12, 16, 20, and 24 weeks after randomization and monthly thereafter until study completion. Each evaluation included measurement of serum LDL levels and recordation of any MACEs or less severe adverse events occurring since the previous evaluation. In addition, certain evaluation periods included measurement of one or more of hs-CRP, sPLA₂, IL-6, and/or other biomarker levels, vital signs, weight, and/or waist circumference. All active subjects (i.e., those who did not have a MACE or withdraw early) received a final evaluation when treatment ended. This final evaluation included at least a complete physical examination, a 12-lead ECG reading, measurement of LDL, hs-CRP, sPLA₂, and IL-6 levels, and recordation of any MACEs during the study period.

The ITT population following randomization contained 313 subjects receiving A-002 plus atorvastatin and 311 subjects receiving atorvastatin only. The number of diabetic subjects in each of these groups was 84 (26.8%) and 87 (28.0%), respectively. Results are set forth in the following Tables.

TABLE 1
Effect of A-002 administration on serum LDL levels in ITT population

| | | | Placebo | |
|---|---|---|---|---|
| | | A-002 plus | (atorvastatin | P-value vs. |
| | | atorvastatin | only) | placebo |
| Week 2 | # of subjects | 182 | 183 | |
| | Change in | −62.4 mg/dl | −52.5 mg/dl | 0.0024 |
| | mean [LDL] | | | |
| | from baseline | | | |
| | % change in | −48.1% | −41.7% | |
| | mean [LDL] | | | |
| | from baseline | | | |
| Week 4 | # of subjects | 250 | 248 | |
| | Change in | −64.2 mg/dl | −55.5 mg/dl | 0.0011 |
| | mean [LDL] | | | |
| | from baseline | | | |
| | % change in | −48.5% | −42.3% | |
| | mean [LDL] | | | |
| | from baseline | | | |
| Week 8 | # of subjects | 246 | 242 | |
| | Change in | −64.6 mg/dl | −56.6 mg/dl | 0.0021 |
| | mean [LDL] | | | |
| | from baseline | | | |
| | % change in | −49.5% | −43.8% | |
| | mean [LDL] | | | |
| | from baseline | | | |
| Week 16 | # of subjects | 235 | 232 | |
| | Change in | −57.9 mg/dl | −48.1 mg/dl | 0.0071 |
| | mean [LDL] | | | |
| | from baseline | | | |
| | % change in | −42.8% | −36.5% | |
| | mean [LDL] | | | |
| | from baseline | | | |
| Week 24 | # of subjects | 179 | 184 | |
| | Change in | −58.1 mg/dl | −50.0 mg/dl | 0.0269 |
| | mean [LDL] | | | |
| | from baseline | | | |
| | % change in | −43.8% | −38.2% | |
| | mean [LDL] | | | |
| | from baseline | | | |

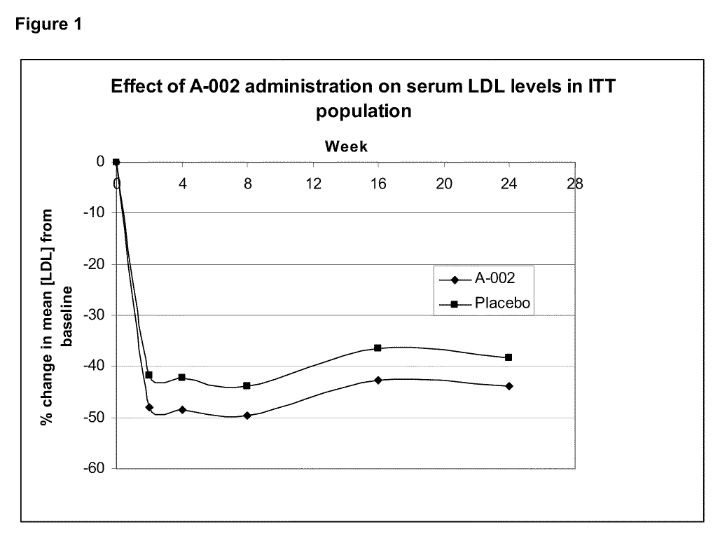

All subject groups exhibited a decrease in mean serum LDL levels at all timepoints measured, which is consistent with the reduction in LDL levels normally seen immediately following an ACS event. Subjects receiving A-002 plus atorvastatin exhibited a greater percent decrease in mean LDL levels at the first timepoint measured (week 2) than subjects receiving atorvastatin alone, and the combination subjects continued to exhibit a greater decrease in LDL levels at all subsequent timepoints. These results, which are further summarized in FIG. 1, show that A-002 in combination with statin reduces LDL levels more rapidly and to a greater extent than statin alone in an unstable post-ACS event population.

TABLE 2
Effect of A-002 administration on reaching target
LDL levels in ITT population

| | | | | Difference |
|---|---|---|---|---|
| | | | | between |
| | | | | A-002/ |
| | | | Placebo | atorvastatin |
| | | A-002 plus | (atorvastatin | and placebo |
| | | atorvastatin | only) | (p-value) |
| Baseline | # of subjects | 266 | 269 | |
| | % of subjects | 24.2% | 24.1% | 0.1% |
| | with [LDL] < | | | |
| | 100 mg/dl | | | |
| | % of subjects | 6.0% | 5.2% | 0.8% |
| | with [LDL] < | | | |
| | 70 mg/dl | | | |
| | % of subjects | 1.5% | 1.1% | 0.4% |
| | with [LDL] < | | | |
| | 50 mg/dl | | | |
| Week 2 | # of subjects | 182 | 183 | |
| | % of subjects | 93.9% | 89.1% | 4.8% |
| | with [LDL] < | | | (0.0802) |
| | 100 mg/dl | | | |
| | % of subjects | 62.6% | 54.6% | 8.0% |
| | with [LDL] < | | | (0.0859) |
| | 70 mg/dl | | | |
| | % of subjects | 32.4% | 21.9% | 10.5% |
| | with [LDL] < | | | (0.0184) |
| | 50 mg/dl | | | |
| Week 4 | # of subjects | 250 | 248 | |
| | % of subjects | 92.8% | 91.2% | 1.6% |
| | with [LDL] < | | | (0.4357) |
| | 100 mg/dl | | | |
| | % of subjects | 70.4% | 54.8% | 15.6% |
| | with [LDL] < | | | (0.0003) |
| | 70 mg/dl | | | |
| | % of subjects | 36.4% | 25.0% | 11.4% |
| | with [LDL] < | | | (0.0061) |
| | 50 mg/dl | | | |
| Week 8 | # of subjects | 246 | 242 | |
| | % of subjects | 92.7% | 91.4% | 1.3% |
| | with [LDL] < | | | (0.7312) |
| | 100 mg/dl | | | |
| | % of subjects | 70.3% | 59.9% | 10.4% |
| | with [LDL] < | | | (0.0106) |
| | 70 mg/dl | | | |
| | % of subjects | 34.1% | 22.7% | 11.4% |
| | with [LDL] < | | | (0.0054) |
| | 50 mg/dl | | | |
| Week 16 | # of subjects | 235 | 232 | |
| | % of subjects | 88.0% | 82.4% | 5.6% |
| | with [LDL] < | | | (0.0982) |
| | 100 mg/dl | | | |
| | % of subjects | 64.3% | 48.3% | 16.0% |
| | with [LDL] < | | | (0.0005) |
| | 70 mg/dl | | | |
| | % of subjects | 30.6% | 16.8% | 13.8% |
| | with [LDL] < | | | (0.0004) |
| | 50 mg/dl | | | |
| Week 24 | # of subjects | 179 | 184 | |
| | % of subjects | 88.7% | 86.5% | 2.2% |
| | with [LDL] < | | | (0.2417) |
| | 100 mg/dl | | | |
| | % of subjects | 61.5% | 50.0% | 11.5% |
| | with [LDL] < | | | (0.0204) |
| | 70 mg/dl | | | |
| | % of subjects | 34.6% | 14.1% | 20.5% |
| | with [LDL] < | | | (<0.0001) |
| | 50 mg/dl | | | |

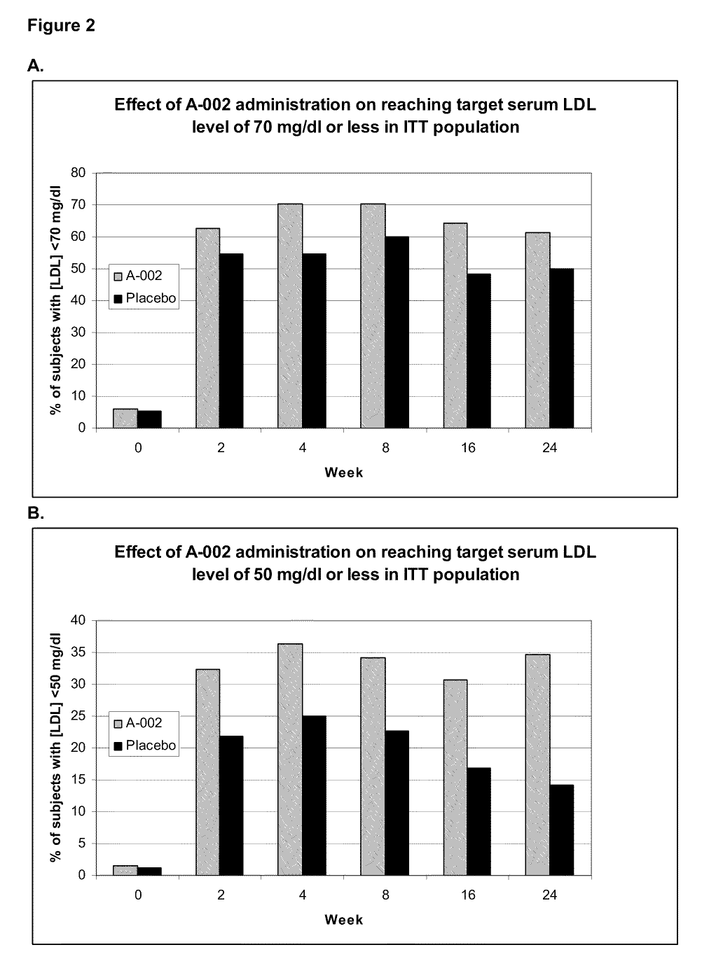

The percentage of subjects achieving target LDL-C levels of 100 mg/dl or less, 70 mg/dl or less, or 50 mg/dl or less was greater in the A-002/atorvastatin group than in the atorvastatin only group at all timepoints. Since atorvastatin alone was fairly effective at reducing LDL levels to 100 mg/dl or below, the difference between the two groups for this target level was relatively small. However, A-002 plus atorvastatin was significantly more effective at reducing LDL levels to 70 mg/dl or less or 50 mg/dl or less target levels than atorvastatin alone. The lower the target LDL-C level, the greater the difference in efficacy between the A-002/atorvastatin group and the placebo group. These results, which are further summarized in FIG. 2, show that the combination of A-002 and atorvastatin is more effective at helping subjects reach specific LDL-C goals than atorvastatin alone. Subjects receiving A-002/atorvastatin combination therapy reach target LDL-C levels more quickly than subjects receiving traditional atorvastatin therapy, and they maintain these decreased LDL levels longer. At the final study visit, the percentage of subjects in the A-002/atorvastatin group reaching target LDL levels of 70 mg/dl or less was 54.9%, versus 42.8% for the atorvastatin only group. Similarly, 24.8% of subjects in the A-002/atorvastatin group reached target LDL levels of 50 mg/dl or less, versus 16.0% of subjects in the atorvastatin only group.

TABLE 3
Effect of A-002 administration on serum hs-CRP levels in ITT population

| | | | | Difference |
|---|---|---|---|---|
| | | | | between |
| | | | | A-002/ |
| | | | Placebo | atorvastatin |
| | | A-002 plus | (atorvastatin | and placebo |
| | | atorvastatin | only) | (p-value) |
| Week 2 | # of subjects | 184 | 183 | |
| | % change in | −40.5% | −20.0% | 20.5% |
| | median [hs- | | | (0.1791) |
| | CRP] from | | | |
| | baseline | | | |
| Week 4 | # of subjects | 259 | 252 | |
| | % change in | −69.2% | −63.5% | 5.7% |
| | median [hs- | | | (0.3976) |
| | CRP] from | | | |
| | baseline | | | |
| Week 8 | # of subjects | 290 | 283 | |
| | % change in | −75.0% | −71.0% | 4.00% |
| | median [hs- | | | (0.0931) |
| | CRP] from | | | |
| | baseline | | | |
| Week 16 | # of subjects | 245 | 239 | |
| | % change in | −81.6% | −71.8% | 9.8% |
| | median | | | (0.0021) |
| | [hs-CRP] | | | |
| | from baseline | | | |
| Week 24 | # of subjects | 188 | 189 | |
| | % change in | −79.8% | −77.0% | 2.8% |
| | log mean | | | (0.0185) |
| | [hs-CRP] | | | |
| | from baseline | | | |
| | % change in | | | |
| | median [hs- | | | |
| | CRP] from | | | |
| | baseline | | | |

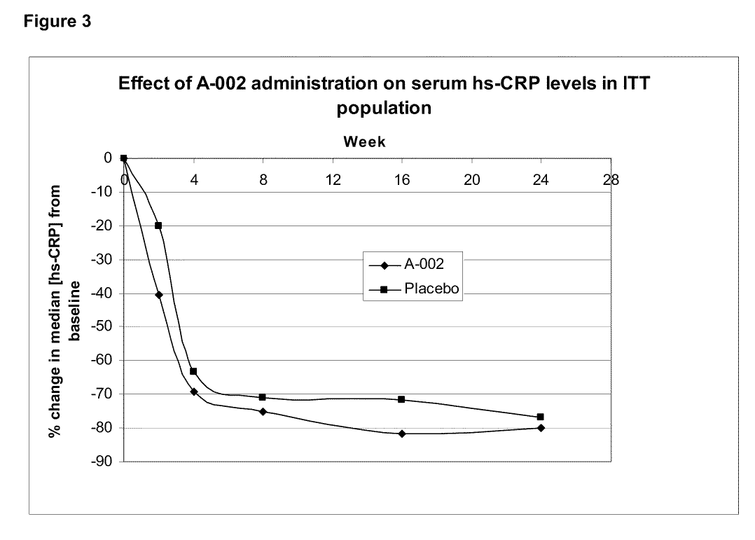

All groups exhibited a decrease in median hs-CRP levels from baseline at all timepoints measured. Subjects in the ITT population receiving A-002 plus atorvastatin exhibited a greater percent decrease in median hs-CRP levels than subjects receiving atorvastatin alone at all timepoints, indicating that A-002 plus statin reduces the post-ACS event inflammatory response to a greater degree than statin alone. This difference was most pronounced at week 2, indicating that A-002 plus statin acts rapidly to reduce inflammation in the critical time period immediately following an ACS event. These results, which are further summarized in FIG. 3, indicate that A-002 plus statin is capable of rapidly reducing inflammation immediately following an ACS event in a population with very high levels of inflammation.

TABLE 4
Effect of A-002 administration on serum hs-CRP levels in diabetes
subpopulation

| | | | | Difference |
|---|---|---|---|---|
| | | | | between A- |
| | | | Placebo | 002/atorvastatin |
| | | A-002 plus | (atorvastatin | and placebo |
| | | atorvastatin | only) | (p-value) |
| Week 2 | # of subjects | 46 | 55 | |
| | % change in | −58.8% | −11.0 | 47.8% |
| | median [hs- | | | (0.0004) |
| | CRP] from | | | |
| | baseline | | | |
| Week 4 | # of subjects | 69 | 70 | |
| | % change in | −83.0% | −51.1% | 31.9% |
| | median [hs- | | | (0.0013) |
| | CRP] from | | | |
| | baseline | | | |
| Week 8 | # of subjects | 70 | 63 | |
| | % change in | −82.8% | −67.6% | 15.2% |
| | median [hs- | | | (0.0299) |
| | CRP] from | | | |
| | baseline | | | |
| Week 16 | # of subjects | 67 | 65 | |
| | % change in | −83.6% | −72.4% | 11.2% |
| | median [hs- | | | (0.0776) |
| | CRP] from | | | |
| | baseline | | | |
| Week 24 | # of subjects | 50 | 53 | |
| | % change in | −89.5% | −76.1% | 13.4% |
| | median [hs- | | | (0.0311) |
| | CRP] from | | | |
| | baseline | | | |

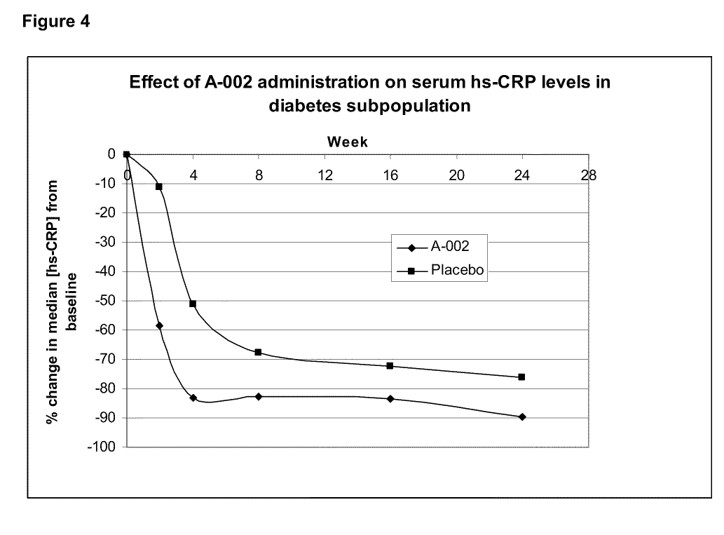

All groups in a diabetic subpopulation exhibited a decrease in median hs-CRP levels from baseline at all timepoints measured. Subjects receiving A-002 plus atorvastatin exhibited a greater percent decrease in median hs-CRP levels than subjects receiving atorvastatin alone at all timepoints. These results, which are further summarized in FIG. 4, indicate that A-002 plus statin is capable of rapidly reducing inflammation immediately following an ACS event in a population with very high levels of inflammation, such as a population with diabetes or metabolic syndrome.

TABLE 5
Effect of A-002 administration on serum sPLA₂ levels in ITT population

| | | | | Difference |
|---|---|---|---|---|
| | | | | between |
| | | | | A-002/ |
| | | | Placebo | atorvastatin |
| | | A-002 plus | (atorvastatin | and placebo |
| | | atorvastatin | only) | (p-value) |
| Week 2 | # of subjects | 133 | 136 | |
| | % change in | −83.8% | +5.4% | 89.2% |
| | median | | | (<0.0001) |
| | [sPLA₂] from | | | |
| | baseline | | | |
| Week 4 | # of subjects | 139 | 145 | |
| | % change in | −80.6% | −7.0% | 73.6% |
| | median | | | (<0.0001) |
| | [sPLA₂] from | | | |
| | baseline | | | |
| Week 8 | # of subjects | 169 | 159 | |
| | % change in | −82.4% | −15.6% | 66.8% |
| | median | | | (<0.0001) |
| | [sPLA₂] from | | | |
| | baseline | | | |
| Week 16 | # of subjects | 49 | 37 | |
| | % change in | −62.0% | −12.1% | 49.9% |
| | median | | | (<0.0001) |
| | [sPLA₂] from | | | |
| | baseline | | | |

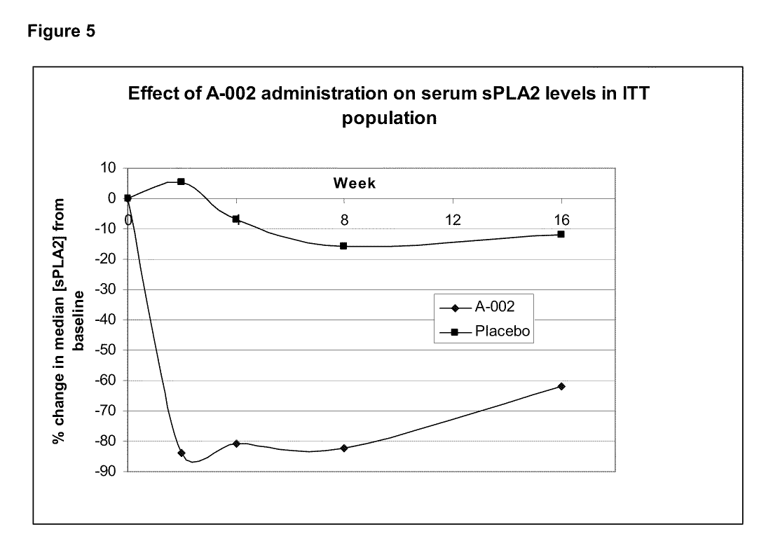

Subjects receiving atorvastatin only exhibited an increase in median sPLA₂ levels at week 2 and a decrease at all other timepoints, while subjects receiving A-002 plus atorvastatin exhibited a decrease in median sPLA₂ levels at all timepoints. The percent decrease in median sPLA₂ levels was significantly greater for subjects receiving A-002 plus atorvastatin versus subjects receiving atorvastatin only at all timepoints. The greatest difference between the A-002/atorvastatin and atorvastatin groups occurred at week 2. These results, which are further summarized in FIG. 5, provide additional confirmation that A-002 plus statin reduces inflammation following an ACS event more quickly and effectively than statin alone.

TABLE 6
Effect of A-002 administration on serum IL-6 levels in ITT population

| | | | | Difference |
|---|---|---|---|---|
| | | | | between A- |
| | | | Placebo | 002/atorvastatin |
| | | A-002 plus | (atorvastatin | and placebo |
| | | atorvastatin | only) | (p-value) |
| Week 2 | # of subjects | 186 | 181 | |
| | % change in | −16.6% | −5.2% | 11.4% |
| | median [IL-6] | | | (0.1924) |
| | from baseline | | | |
| Week 4 | # of subjects | 212 | 202 | |
| | % change in | −41.3% | −33.7% | 7.6% |
| | median [IL-6] | | | (0.3397) |
| | from baseline | | | |
| Week 8 | # of subjects | 170 | 159 | |
| | % change in | −42.4% | −38.3% | 4.0% |
| | median [IL-6] | | | (0.5613) |
| | from baseline | | | |

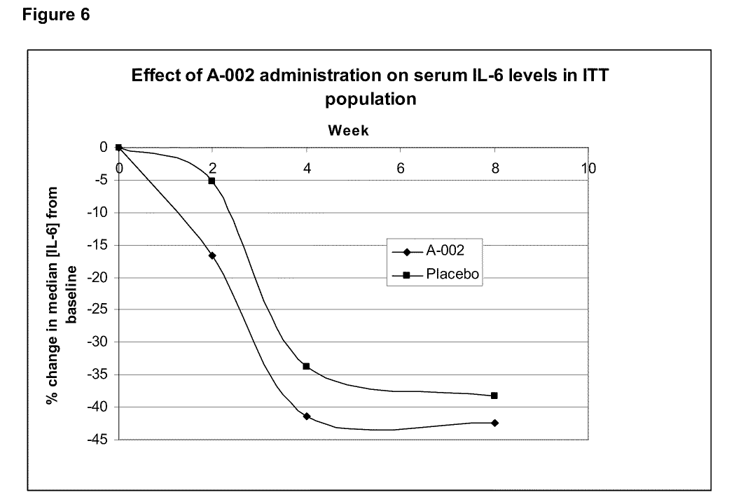

All groups exhibited a decrease in median IL-6 levels from baseline at all timepoints measured. Subjects in the ITT population receiving A-002 plus atorvastatin exhibited a greater percent decrease in median IL-6 levels than subjects receiving atorvastatin alone at all timepoints. This difference was most pronounced at week 2. These results, which are further summarized in FIG. 6, further confirm that A-002 plus statin reduces the post-ACS event inflammatory response to a greater degree than statin alone, particularly during the first four weeks after an ACS event when the risk is highest.

TABLE 7
Effect of A-002 administration on serum IL-6 levels
in diabetes subpopulation

| | | | | Difference |
|---|---|---|---|---|
| | | | | between A- |
| | | | Placebo | 002/atorvastatin |
| | | A-002 plus | (atorvastatin | and placebo |
| | | atorvastatin | only) | (p-value) |
| Week 2 | # of subjects | 48 | 54 | |
| | % change in | −21.8% | +3.5% | 25.4% |
| | median [IL-6] | | | (0.0019) |
| | from baseline | | | |
| Week 4 | # of subjects | 55 | 59 | |
| | % change in | −47.1% | −23.9% | 13.2% |
| | median [IL-6] | | | (0.0250) |
| | from baseline | | | |
| Week 8 | # of subjects | 51 | 51 | |
| | % change in | −48.8% | −40.4% | 8.8% |
| | median [IL-6] | | | (0.0903) |
| | from baseline | | | |

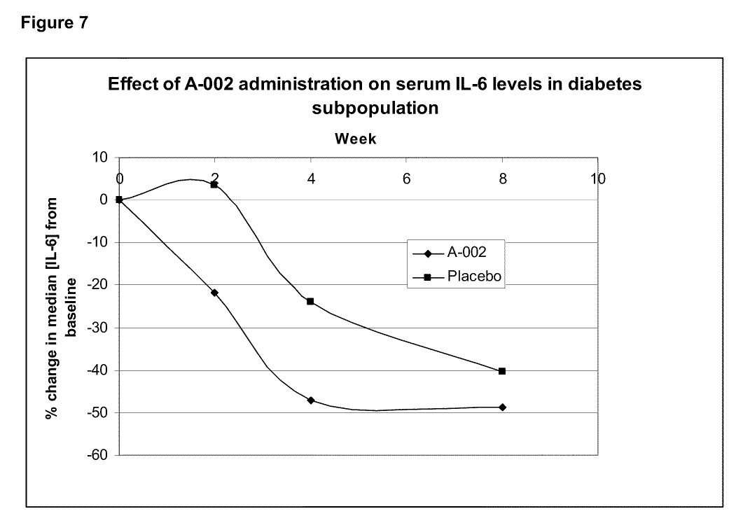

Diabetic subjects receiving atorvastatin alone exhibited an increase in median IL-6 levels from baseline at week 2, followed by a decrease at weeks 4 and 8. Subjects receiving A-002 plus atorvastatin exhibited a decrease in median IL-6 levels from baseline at all timepoints. The greatest difference between subjects receiving A-002 plus atorvastatin versus subjects receiving atorvastatin alone was seen at weeks 2 and 4. These results, which are further summarized in FIG. 7, confirm that A-002 plus statin is capable of rapidly reducing inflammation after an ACS event in a population with very high levels of inflammation, such as a population with diabetics or metabolic syndrome.

TABLE 8
Effect of A-002 administration on reaching target LDL and
CRP levels in ITT population

| | | | | Difference |
|---|---|---|---|---|
| | | | | between |
| | | | | A-002/ |
| | | | Placebo | atorvastatin |
| | | A-002 plus | (atorvastatin | and placebo |
| | | atorvastatin | only) | (p-value) |
| Base- | # of subjects | 277 | 282 | — |
| line | % of subjects | 2.2% | 1.4% | 0.8% |
| | with mean | | | |
| | [LDL] < 70 mg/dl | | | |
| | and [hs- | | | |
| | CRP] < 3 mg/L | | | |
| | % of subjects | 1.1% | 0.4% | 0.7% |
| | with mean | | | |
| | [LDL] < 70 mg/dl | | | |
| | and [hs- | | | |
| | CRP] <1 mg/L | | | |
| Week 2 | # of subjects | 172 | 177 | |
| | % of subjects | 24.4% | 16.4% | 8.0% |
| | with mean | | | (0.0623) |
| | [LDL] < 70 mg/dl | | | |
| | and [hs- | | | |
| | CRP] < 3 mg/L | | | |
| | % of subjects | 8.1% | 5.6% | 2.5% |
| | with mean | | | (0.3581) |
| | [LDL] < 70 mg/dl | | | |
| | and [hs- | | | |
| | CRP] <1 mg/L | | | |
| Week 4 | # of subjects | 243 | 243 | |
| | % of subjects | 45.3% | 30.5% | 14.8% |
| | with mean | | | (0.0008) |
| | [LDL] < 70 mg/dl | | | |
| | and [hs- | | | |
| | CRP] < 3 mg/L | | | |
| | % of subjects | 20.6% | 12.8% | 7.8% |
| | with mean | | | (0.0207) |
| | [LDL] < 70 mg/dl | | | |
| | and [hs- | | | |
| | CRP] <1 mg/L | | | |
| Week 8 | # of subjects | 238 | 234 | |
| | % of subjects | 47.1% | 36.8% | 10.3% |
| | with mean | | | (0.0233) |
| | [LDL] < 70 mg/dl | | | |
| | and [hs- | | | |
| | CRP] < 3 mg/L | | | |
| | % of subjects | 19.3% | 17.5% | 1.8% |
| | with mean | | | (0.6128) |
| | [LDL] < 70 mg/dl | | | |
| | and [hs- | | | |
| | CRP] <1 mg/L | | | |
| Week | # of subjects | 229 | 231 | |
| 16 | % of subjects | 50.7% | 32.9% | 17.8% |
| | with mean | | | (0.0001) |
| | [LDL] < 70 mg/dl | | | |
| | and [hs- | | | |
| | CRP] < 3 mg/L | | | |
| | % of subjects | 24.9% | 15.6% | 9.3% |
| | with mean | | | (0.0130) |
| | [LDL] < 70 mg/dl | | | |
| | and [hs- | | | |
| | CRP] < 1 mg/L | | | |
| Week | # of subjects | 175 | 182 | |
| 24 | % of subjects | 50.9% | 34.1% | 16.8% |
| | with mean | | | (0.0013) |
| | [LDL] < 70 mg/dl | | | |
| | and [hs- | | | |
| | CRP] < 3 mg/L | | | |
| | % of subjects | 26.9% | 15.9% | 11.0% |
| | with mean | | | (0.0117) |
| | [LDL] < 70 mg/dl | | | |
| | and [hs- | | | |
| | CRP] < 1 mg/L | | | |

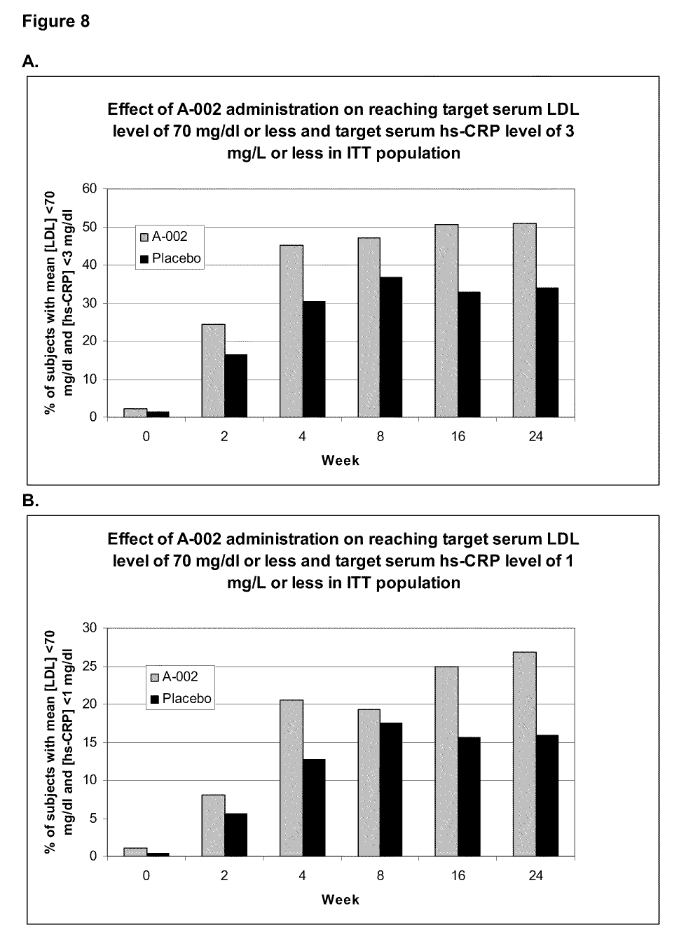

The percentage of subjects achieving a composite target of less than 70 mg/dl LDL and less than 3 mg/L hs-CRP or less than 70 mg/dl and less than 1 mg/L hs-CRP was greater in the A-002/atorvastatin group than in the atorvastatin only group at all timepoints. These results, which are further summarized in FIG. 8, further confirm that A-002 plus atorvastatin is more effective than statin alone at reducing LDL levels and inflammation in the period immediately following an ACS event.

Given the ability of A-002 plus atorvastatin to decrease LDL levels and inflammation to a greater degree than atorvastatin alone in the period following an ACS event, it was expected that co-administration of A-002 and atorvastatin would decrease the occurrence of one or more MACEs. The effect of A-002 and atorvastatin administration on MACEs is summarized in the following Tables.

TABLE 9
Effect of A-002 administration on specific MACE subtypes at
various time intervals

| | | | | A-002/ |
|---|---|---|---|---|
| | | | | atorvastatin |
| | | | | group vs. |
| | | A-002/ | Placebo | placebo group |
| | | atorvastatin | (atorvastatin only) | (% reduction in |
| | | (# of events) | (# of events) | events) |
| N in ITT population | | 313 | 311 | |
| 0-30 days | UA | 4 | 6 | 33% |
| | MI | 2 | 2 | — |
| | Death | 3 | 4 | 25% |
| | Stroke | 1 | 0 | — |
| | Total | 10 | 12 | 17% |
| 30-60 days | UA | 1 | 1 | — |
| | MI | 0 | 1 | 100% |
| | Death | 0 | 0 | — |
| | Stroke | 0 | 0 | — |
| | Total | 1 | 2 | 50% |
| 60-90 days | UA | 0 | 1 | 100% |
| | MI | 0 | 0 | — |
| | Death | 2 | 1 | — |
| | Stroke | 0 | 1 | 100% |
| | Total | 2 | 3 | 33% |
| 90-112 days | UA | 0 | 1 | 100% |
| | MI | 0 | 1 | 100% |
| | Death | 0 | 0 | — |
| | Stroke | 0 | 0 | — |
| | Total | 0 | 2 | 100% |

TABLE 10
Cumulative effect of A-002 administration on specific MACE subtypes
at various time intervals

| | | | | Reduction in A- |
|---|---|---|---|---|
| | | | | 002/atorvastatin |
| | | | Placebo | group vs. |
| | | A-002/ | (atorvastatin | placebo group |
| | | atorvastatin | only) | (% reduction in |
| | | (# of events) | (# of events) | events) |
| N in ITT population | | 313 | 311 | |
| 0-30 days | UA | 4 | 6 | 33% |
| | MI | 2 | 2 | — |
| | Death | 3 | 4 | 25% |
| | Stroke | 1 | 0 | — |
| | Total | 10 | 12 | 17% |
| 0-60 days | UA | 5 | 7 | 29% |
| | MI | 2 | 3 | 33% |
| | Death | 3 | 4 | 25% |
| | Stroke | 1 | 0 | — |
| | Total | 11 | 14 | 21% |
| 0-90 days | UA | 5 | 8 | 38% |
| | MI | 2 | 3 | 33% |
| | Death | 5 | 5 | — |
| | Stroke | 1 | 1 | — |
| | Total | 13 | 17 | 24% |
| 0-112 days | UA | 5 | 9 | 44% |
| | MI | 2 | 4 | 50% |
| | Death | 5 | 5 | — |
| | Stroke | 1 | 1 | — |
| | Total | 13 | 19 | 32% |

TABLE 11
Cumulative effect of A-002 administration on MACEs at 16 weeks

| | A-002 plus | Placebo |
|---|---|---|
| | atorvastatin | (atorvastatin only) |
| ITT population (n) | 313 | 311 |
| Total MACEs | 13 (4.2%) | 19 (6.1%) |
| UA requiring | 5 (1.6%) | 9 (2.9%) |
| hospitalization | | |
| MI | 2 (0.6%) | 4 (1.3%) |
| Stroke | 1 (0.3%) | 1 (0.3%) |
| Death | 5 (1.6%) | 5 (1.6%) |
| Revascularization ≧ 60 | 0 | 0 |
| days | | |

Subjects receiving A-002 plus atorvastatin experienced fewer MACEs than subjects receiving atorvastatin during each interval from days 0 to 30, days 30 to 60, days 60 to 90, and days 90 to 112. At 16 weeks, 13 out of 313 subjects (4.2%) receiving A-002 plus atorvastatin had experienced a MACE, versus 19 out of 311 subjects (6.1%) in the atorvastatin only population. These results show that administration of A-002 plus atorvastatin significantly reduces the likelihood of experiencing a MACE in the 112 days (16 weeks) following an index ACS event.

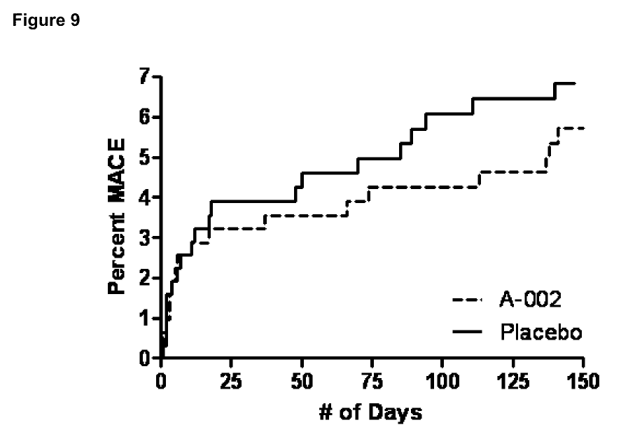

The decrease in MACE occurrence in the A-002/atorvastatin group versus the placebo group was not limited to one MACE type, as decreases were observed in both UA and MI at 16 weeks. A decrease in deaths was also observed during the first 60 days following the ACS event. Only one subject from either group experienced a stroke during the course of the trial, giving insufficient data for meaningful statistical analysis. As expected with an unstable population that had recently experienced an ACS event, the majority of MACEs during the trial occurred during the earlier timepoints. A-002 plus atorvastatin reduced MACEs during this critical period to a greater extent than atorvastatin alone. From days 0 to 30, 10 out of 313 subjects (3.2%) in the A-002/atorvastatin group experienced a MACE, as compared to 12 out of 311 (3.9%) in the atorvastatin only group. From days 0 to 60, 11 out of 313 subjects (3.5%) in the A-002/atorvastatin group experienced a MACE, as compared to 14 out of 311 (4.5%) in the atorvastatin only group. From days 0 to 90, 13 of 313 subjects (4.2%) in the A-002/atorvastatin group experienced a MACE, as compared to 17 out of 311 subjects (5.5%) in the atorvastatin only group. Although the effect was most pronounced in the initial 16 weeks following an index ACS event, subjects receiving A-002 plus atorvastatin continued to exhibit a decrease in MACEs versus subjects receiving atorvastatin alone all the way out to the end of the trial (150 days) (FIG. 9).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Boekholdt et al. 2005. Arterioscler Thromb Vasc Biol 25:839-846.
2. Bostrom et al. 2007. Arterioscler Thromb Vasc Biol 27:600-606.
3. Camejo et al. 1998. Atherosclerosis 139:205-222.
4. Cannon et al. 2004. N Engl J Med 350:1495.
5. Chait, A., et al. 2005. J Lipid Res 46:389-403.
6. Elinder et al. 1997. Arterioscler Thromb Vasc Biol 17:2257-2263.
7. Hakala et al. 2001. Arterioscler Thromb Vasc Biol 21:1053-1058.
8. Hartford et al. 2006. J Cardiol 108:55-62.
9. Hurt-Camejo et al. 1997. Arterioscler Thromb Vasc Biol 17:300-309.
10. Ivandic et al. 1999. Arterioscler Thromb Vasc Biol 19:1284-1290.
11. Kimura-Matsumoto et al. 2008. Atherosclerosis 196:81-91.
12. Kugiyama et al. 1999. Circulation 100:1280-1284.
13. Liu et al. 2003. Eur Heart J 24:1824-1832.
14. Mallat et al. 2005. J Am Coll Cardiol 46:1249-1257.
15. Mallat et al. 2007. Arterioscler Thromb Vasc Biol 27:1177-1183.
16. Menschikowski et al. 1995. Atherosclerosis 118:173-181.
17. Nijmeijer et al. 2002. Cardiovasc Res 53:138-146.
18. Pruzanski et al. 1998. J Lipid Res 39:2150-2160.
19. Ridker et al. 2005. N Engl J Med 352:20-28.
20. Ridker et al. 2008. N Engl J Med 359:2195.
21. Rosengren, B., et al. 2006. Arterioscler Thromb Vasc Biol 26:1579-1585.
22. Sartipy et al. 1999. J Biol Chem 274:25913-25920.
23. Schwartz et al. 2001. JAMA 285:1711-1718.
24. Schwartz & Olsson. 2005. Am J Cardiol 96:45F-53F.
25. Serruys et al. 2008. Circulation 118:1172-1182.
26. Tietge et al. 2000. J Biol Chem 275:10077-10084.

What is claimed is:

1. A method of decreasing the likelihood of a major adverse cardiac event (MACE) in a subject that has previously experienced an acute coronary syndrome (ACS) event comprising administering a therapeutically effective amount of one or more sPLA₂ inhibitors and a therapeutically effective amount of one or more statins to said subject.

2. The method of claim 1, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

3. The method of claim 1, wherein the first administration of said one or more sPLA₂ inhibitors takes place within 96 hours of the occurrence or diagnosis of said ACS event.

4. The method of claim 1, wherein said administration of one or more sPLA₂ inhibitors occurs one or more times daily for a maximum of 16 weeks.

5. The method of claim 2, wherein said prodrug is selected from the group consisting of a C₁-C₆ alkyl ester prodrug, an acyloxyalkyl ester prodrug, and an alkyloxycarbonyloxyalkyl ester prodrug.

6. The method of claim 5, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

7. The method of claim 1, wherein said one or more statins are selected from the group consisting of atorvastatin, rosuvastatin, simvastatin, lovastatin, pravastatin, cerivastatin, fluvastatin, mevastatin, and pitavastatin, and a statin combination drug.

8. The method of claim 1, wherein said MACE is selected from one or more of the group consisting of cardiovascular death, non-fatal myocardial infarction, non-fatal stroke, or unstable angina requiring urgent hospitalization.

9. A method of inhibiting inflammation in a subject that has previously experienced an acute coronary syndrome (ACS) event comprising administering a therapeutically effective amount of one or more sPLA₂ inhibitors and a therapeutically effective amount of one or more statins to said subject.

10. The method of claim 9, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

11. The method of claim 9, wherein the first administration of said one or more sPLA₂ inhibitors takes place within 96 hours of the occurrence or diagnosis of said ACS event.

12. The method of claim 9, wherein said administration of one or more sPLA₂ inhibitors occurs one or more times daily for a maximum of 16 weeks.

13. The method of claim 10, wherein said prodrug is selected from the group consisting of a C₁-C₆ alkyl ester prodrug, an acyloxyalkyl ester prodrug, and an alkyloxycarbonyloxyalkyl ester prodrug.

14. The method of claim 13, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

15. The method of claim 9, wherein said one or more statins are selected from the group consisting of atorvastatin, rosuvastatin, simvastatin, lovastatin, pravastatin, cerivastatin, fluvastatin, mevastatin, and pitavastatin, and a statin combination drug.

16. The method of claim 9, wherein said administration of one or more sPLA₂ inhibitors and one or more statins results in a decrease in one or more inflammatory markers selected from the group consisting of sPLA₂, hs-CRP, and IL-6.

17. A method of lowering non-HDL cholesterol levels in a subject who has previously experienced an acute coronary syndrome (ACS) event comprising administering to said subject a therapeutically effective amount of one or more sPLA₂ inhibitors and a therapeutically effective amount of one or more statins.

18. The method of claim 17, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

19. The method of claim 17, wherein the first administration of said one or more sPLA₂ inhibitors takes place within 96 hours of the occurrence or diagnosis of said ACS event.

20. The method of claim 17, wherein said administration of one or more sPLA₂ inhibitors occurs one or more times daily for a maximum of 16 weeks.

21. The method of claim 18, wherein said prodrug is selected from the group consisting of a C₁-C₆ alkyl ester prodrug, an acyloxyalkyl ester prodrug, and an alkyloxycarbonyloxyalkyl ester prodrug.

22. The method of claim 21, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

23. The method of claim 17, wherein said one or more statins are selected from the group consisting of atorvastatin, rosuvastatin, simvastatin, lovastatin, pravastatin, cerivastatin, fluvastatin, mevastatin, and pitavastatin, and a statin combination drug.

24. The method of claim 17, wherein said administration of one or more sPLA₂ inhibitors and one or more statins results in a decrease in LDL levels.

25. The method of claim 24, wherein LDL levels are decreased to a target level selected from the group consisting of 100 mg/dl or less, 70 mg/dl or less, and 50 mg/dl or less.

26. A method of treating a major adverse cardiac event (MACE) in a subject who has previously experienced an acute coronary syndrome (ACS) event comprising administering a therapeutically effective amount of one or more sPLA₂ inhibitors.

27. The method of claim 26, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

28. The method of claim 27, wherein said prodrug is selected from the group consisting of a C₁-C₆ alkyl ester prodrug, an acyloxyalkyl ester prodrug, and an alkyloxycarbonyloxyalkyl ester prodrug.

29. The method of claim 28, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

30. A method of treating acute coronary syndrome (ACS) in a subject in need thereof comprising administering a therapeutically effective amount of one or more sPLA₂ inhibitors.

31. The method of claim 30, wherein said subject has previously experienced an ACS event.

32. The method of claim 30, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

33. The method of claim 32, wherein said prodrug is selected from the group consisting of a C₁-C₆ alkyl ester prodrug, an acyloxyalkyl ester prodrug, and an alkyloxycarbonyloxyalkyl ester prodrug.

34. The method of claim 33, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

35. A method of treating a major adverse cardiac event (MACE) in a subject who has previously experienced an acute coronary syndrome (ACS) event comprising administering a therapeutically effective amount of one or more sPLA₂ inhibitors and a therapeutically effective amount of one or more statins.

36. The method of claim 35, wherein treatment of MACE results in a decrease in the likelihood of MACE occurrence.

37. The method of claim 35, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

38. The method of claim 37, wherein said prodrug is selected from the group consisting of a C₁-C₆ alkyl ester prodrug, an acyloxyalkyl ester prodrug, and an alkyloxycarbonyloxyalkyl ester prodrug.

39. The method of claim 38, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

40. A method of treating acute coronary syndrome (ACS) in a subject in need thereof comprising administering a therapeutically effective amount of one or more sPLA₂ inhibitors and a therapeutically effective amount of one or more statins.

41. The method of claim 40, wherein said subject has previously experienced an ACS event.

42. The method of claim 40, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

43. The method of claim 42, wherein said prodrug is selected from the group consisting of a C₁-C₆ alkyl ester prodrug, an acyloxyalkyl ester prodrug, and an alkyloxycarbonyloxyalkyl ester prodrug.

44. The method of claim 43, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

45. A method of inhibiting inflammation in a subject who has previously experienced an acute coronary syndrome (ACS) event comprising administering to said subject a therapeutically effective amount of one or more sPLA₂ inhibitors.

46. The method of claim 45, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

47. The method of claim 46, wherein said prodrug is selected from the group consisting of a C₁-C₆ alkyl ester prodrug, an acyloxyalkyl ester prodrug, and an alkyloxycarbonyloxyalkyl ester prodrug.

48. The method of claim 47, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

49. A method of lowering non-HDL cholesterol levels in a subject who has previously experienced an acute coronary syndrome (ACS) event comprising administering to said subject a therapeutically effective amount of one or more sPLA₂ inhibitors.

50. The method of claim 49, wherein said one or more sPLA₂ inhibitors comprise 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof.

51. The method of claim 50, wherein said prodrug is selected from the group consisting of a C₁-C₆ alkyl ester prodrug, an acyloxyalkyl ester prodrug, and an alkyloxycarbonyloxyalkyl ester prodrug.

52. The method of claim 51, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

53. A method of reducing the risk of a major adverse cardiac event (MACE) selected from the group consisting of death, myocardial infarction, stroke, and unstable angina requiring urgent hospitalization in a subject who has experienced or been diagnosed with an acute coronary syndrome (ACS) event within the past 96 hours comprising administering a therapeutically effective amount of 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof and a therapeutically effective amount of one or more statins, wherein 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof is administered once or twice daily for a maximum of 16 weeks.
